(19) United States
(12) Patent Application Publication
(10) Pub. No.: US 2021/0101879 A1
(43) Pub. Date: Apr. 8, 2021

(54) AMYLOID PROTEIN-SELECTIVE BACE INHIBITORS (ASBIs) FOR ALZHEIMER?S DISEASE

(71) Applicant: The Regents of the University of California, Oakland, CA (US)

(72) Inventors: John Varghese, Los Angeles, CA (US); Barbara Jagodzinska, Redwood City, CA (US); Jesus Campagna, Playa Del Rey, CA (US)

(21) Appl. No.: 16/977,341

(22) PCT Filed: Feb. 28, 2019
(86) PCT No.: PCT/US2019/020125

§ 371 Date: Sep. 1, 2020

Related U.S. Application Data
(60) Provisional application No. 62/640,912, filed on Mar. 9, 2018.
(60) Provisional application No. 62/636,952, filed on Mar. 1, 2018.

(51) Int. Cl. C07D 401/04; C07D 233/46; C07D 401/10; C07D 403/04; C07D 413/04; C07D 413/10; C07D 403/10; C07D 413/08; C07D 405/10; C07D 405/14; C07D 401/14

(52) U.S. Cl. CPC C07D 401/04; C07D 233/46; C07D 401/10; C07D 403/04; C07D 413/04; C07D 401/14; C07D 403/10; C07D 413/08; C07D 405/10; C07D 405/14; C07D 413/10

(57) ABSTRACT

The present disclosure provides various compounds, compositions, and methods of BACE inhibition that interact with both BACE and APP to increase selectivity of the inhibitor. Compounds presented herein exhibit desirably low IC₅₀ values and permeability across the blood-brain barrier.

### RELATED APPLICATIONS

This application claims the benefit of priority to U.S. Provisional Application Nos. 62/636,952, filed Mar. 1, 2018 and 62/640,912, filed Mar. 9, 2018, the contents of each of which are incorporated herein by reference in their entirety.

### BACKGROUND OF THE INVENTION

The brain tissue of Alzheimer's disease (AD) patients is characterized by the presence of amyloid plaques largely comprised of amyloid-β (Aβ) peptide [Hardy & Allsop, Trends Pharmacol Sci. 1991, 12:10]. An increase in Aβ production, and/or a decrease in clearance is reported to be a key pathological mechanism in AD. Aβ is generated by sequential cleavage of parent molecule full-length amyloid precursor protein (FL APP) by the aspartyl protease BACE1 (β-site APP cleaving enzyme, BACE) and the γ secretase complex. Because BACE cleavage is the first and rate-limiting step in Aβ production, it has been a target for AD therapeutic development.

Direct inhibition of BACE activity has a risk, however, of leading to inhibition of cleavage of non-APP substrates such as neuregulin-1 (NRG-1). Inhibition of BACE1 cleavage of NRG1 can impair cognitive function. In addition, inhibition of enzymatic activity of non-BACE1 enzymes, particularly BACE2 or cathepsin D (Cat D), is also a potential problem in development of a BACE inhibitor. An APP-Selective BACE Inhibitor (or ASBI) would be selective for both substrate and enzyme and thus be less likely to have adverse side effects.

### SUMMARY OF THE DISCLOSURE

The present disclosure provides a compound of formula (I):

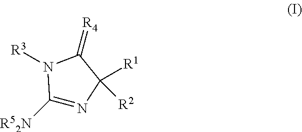

wherein:
R¹ is alkyl, heteroalkyl, cycloalkyl, heterocyclyl, heterocyclylalkyl, aryl, aralkyl, heteroaryl, heteroarylalkyl, aryl-aryl, aryl-heteroaryl, NR³(CO)R⁶, OR⁶ or CO₂R⁶;
R² is alkyl, heteroalkyl, cycloalkyl, heterocyclyl, heterocyclylalkyl, aryl, aralkyl, heteroaryl, heteroarylalkyl, OR⁶, CO₂R⁶, NR³(CO)R⁶, or (CO)NR³R⁶wherein R³ and R⁶, taken together with the N to which they are attached, may form a heterocyclyl or heteroaryl moiety;
each R³ is independently hydrogen, alkyl, aryl or aralkyl;
R⁴ is O, S or NR³;
each R⁵ is independently H or alkyl;
each R⁶ is independently H, alkyl, heteroalkyl, cycloalkyl, heterocyclyl, heterocyclylalkyl, aryl, aralkyl, heteroaryl, heteroarylalkyl, alkoxy or fluoroalkoxy;
or a pharmaceutically acceptable salt thereof.

In certain aspects, the present disclosure provides pharmaceutical formulations comprising a compound of formula I.

In some aspects, the present disclosure provides methods of treating Alzheimer's disease, comprising administering a compound formula I or a pharmaceutical composition comprising a compound of formula Ito a patient in need thereof.

These and other aspects will be described more fully herein.

### BRIEF DESCRIPTION OF THE DRAWINGS

FIG. 1 is a graph showing BACE 1 activity for representative ASBI compounds in a cell-free BACE enzyme assay.

FIG. 2 is a graph showing representative ASBI dose-response inhibition of BACE 1 activity in a cell-free assay utilizing a P5-P5′ substrate.

FIGS. 3A-B are graphs of data from an in vivo assay with Chinese hamster ovary cells stably transfected with human amyloid precursor protein wildtype (CHO-7W) in culture, showing that treatment for 24 hours with FAH-65 (and FAHs 43, 57, and 66) significantly decreased the product of BACE cleavage of APP, soluble amyloid precursor protein beta (sAPPβ, FIG. 3A) and BACE1 and gamma secretase cleavage product amyloid-beta (Aβ, FIG. 3B).

FIGS. 4A-C are graphs showing inhibitory activity of FAH-65 in cleavage of neuregulin 1 (NRG-1, FIG. 4A), or p-selectin glycoprotein ligand 1 (PSGL-1, FIG. 4B), or NRG-1 BACE (FIG. 4C). FAH-65 exhibited little cleavage, indicating FAH-65 is selective for substrate.

FIG. 5 is a graph showing the IC₅₀ for FAH-65 for cathepsin D (˜25 μM), whereas it is 0.005-0.01 μM for BACE1, indicating it is highly selective for inhibition of BACE.

FIGS. 6A-B are graphs showing pharmacokinetics in mice of FAH-65 for oral and SQ administration (FIG. 6A). FAH-65 was found to be brain-penetrant in mice after oral delivery at 30 mg/kg, showing a peak in brain of 74 ng/g at one hour post-dosing (FIG. 6B).

FIGS. 7A-B are graphs showing pilot study results in AD model mice. FAH-65 delivered orally at 30 mg/kg/day for 10 days, showed target engagement, decreasing APP-derived BACE cleavage product βCTF (beta C-terminal fragment) significantly (FIG. 7A), and showed a trend to decrease BACE/γ-secretase product Aβ (FIG. 7B).

FIG. 8 is a graph showing the results of FAH-65 pharmacokinetic and pharmacodynamic studies in rats. The pilot study of FAH-65 effects in comparison to verubecestat was performed using wildtype Sprague-Dawley rats wherein they received 30 mg/kg FAH-65 or verubecestat orally. The brain peak for FAH-65 was 182 ng/g at 2 hours.

FIGS. 9A-B are graphs showing results of the pilot study in rats. CSF (cerebrospinal fluid) and brain tissue were collected 1, 2, 3, 6 12, and 24 hours after dosing, with one rat for each time point. Untreated rats were used as controls. FAH-65 reduced sAPPβ at 3 and 6 hours in CSF (1- and 2-hour sample were not in sufficient volume for assay) (FIG. 9A). Aβ1-40 levels were also decreased by FAH-65 as compared to control, and the decrease persisted to 24 hours (FIG. 9B).

FIGS. 10A-C are graphs showing FAH-65 enantiomer and racemate results in the P5-P5′ assay (FIG. 10A). Enantiomeric peak 1 showed dose-response BACE inhibition whereas peak 2 did not. In CHO-7W cells, FAH-65 peak 1 showed a dose-response inhibition of production of sAPPβ (FIG. 10B) and Aβ (FIG. 10C).

FIG. 11 is graph showing FAH-127 inhibition of BACE. Performance of FAH-127 is similar to FAH-65, with an IC₅₀ of 13 nM.

FIG. 12 is a graph showing pharmacokinetics of FAH-127. FAH-127 shows good brain-penetrance in mice, reaching a peak of ˜3000 (range 600-6000) ng/g at 1 hour.

FIG. 13 is a graph showing P5-P5′ assay data for FAH-65, FAH-65 Peak 1 (enantiomer-1) and FAH-65 Peak 2 (enantiomer-2).

FIG. 14 is a graph showing CatD assay data for FAH-65, FAH-65 Peak 1 (enantiomer-1) and FAH-65 Peak 2 (enantiomer-2). Compounds were tested at 20 μM.

FIG. 15 is a graph showing sAPPβ assay data for FAH-65, FAH-65 Peak 1 and FAH-65 Peak 2. Compounds were tested in CHO-7W cells.

FIG. 16 is a graph showing Aβ1-42 assay data for FAH-65, FAH-65 Peak 1 and FAH-65 Peak 2. Compounds were tested in CHO-7W cells.

FIG. 17 is a graph showing sAPPα assay data for FAH-65, FAH-65 Peak 1 and FAH-65 Peak 2. Compounds were tested in CHO-7W cells.

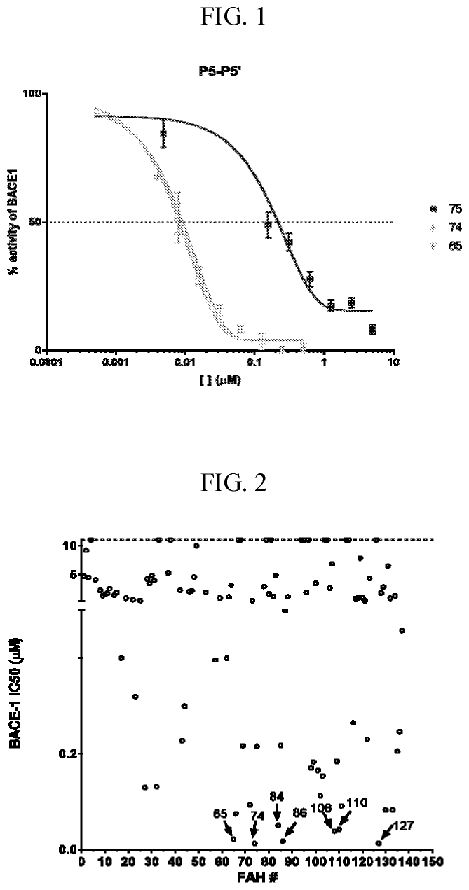

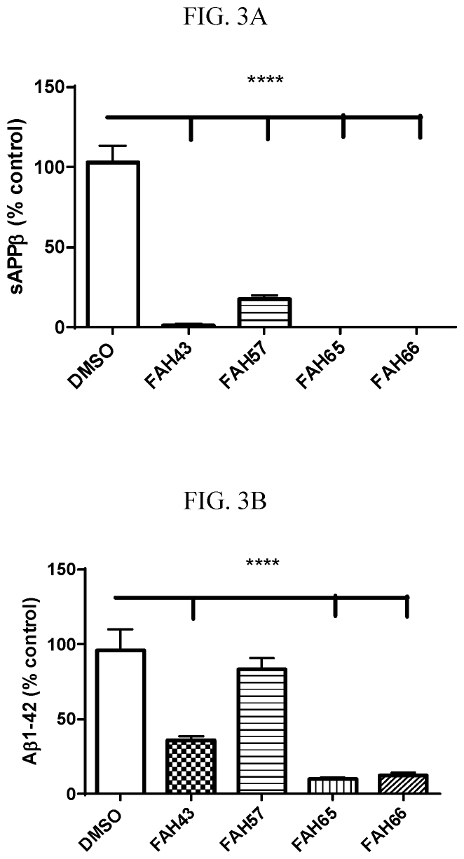

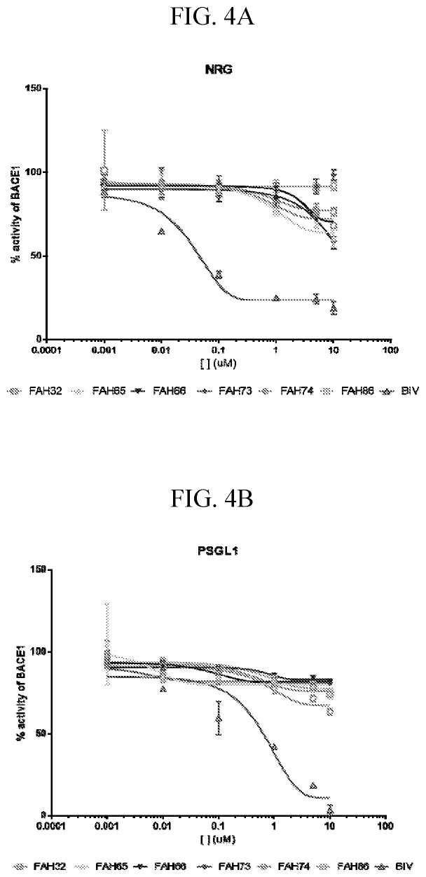

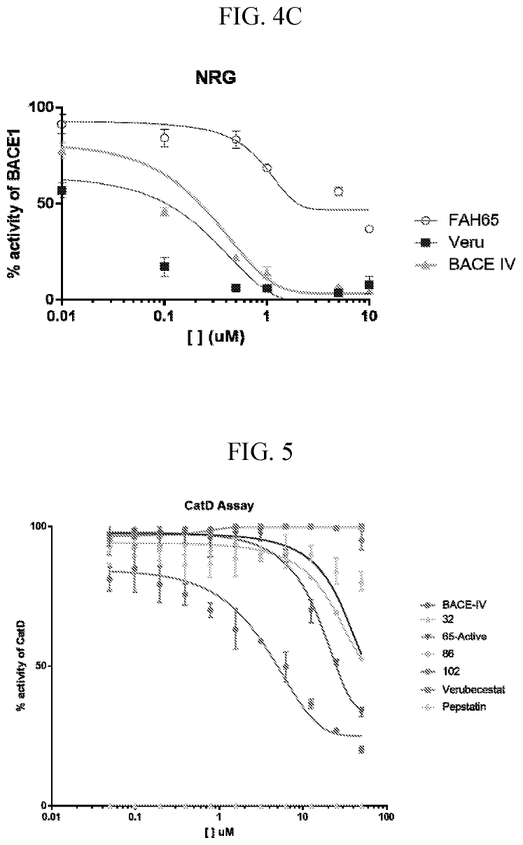

### DETAILED DESCRIPTION OF THE INVENTION

The present disclosure provides fluoroamino hydantoin (FAH) compounds selective for BACE 1 and FL APP inhibition. In an initial screen, it was found that such fluoroamino hydantoin compounds were selective for both BACE1 as an enzyme, showing low/no inhibition of BACE2 or Cat D, and FL APP as the substrate as compared to NRG-1 or p-selectin glycoprotein ligand 1 (PSGL-1). In addition, it was determined by surface plasmon resonance (SPR) analysis to interact directly with both APP and BACE.

The present disclosure provides various compounds, compositions, and methods of BACE inhibition that interact with both BACE and APP to increase selectivity of the inhibitor. Compounds presented herein exhibit suitable IC₅₀ values and permeability across the BBB.

### Compounds

In one aspect, the present disclosure provides a compound of formula (I):

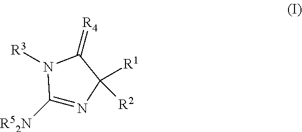

or a pharmaceutically acceptable salt thereof, wherein:
R¹ is alkyl, heteroalkyl, cycloalkyl, heterocyclyl, heterocyclylalkyl, aryl, aralkyl, heteroaryl, heteroarylalkyl, aryl-aryl, aryl-heteroaryl, NR³(CO)R⁶, OR⁶ or CO₂R⁶;
R² is alkyl, heteroalkyl, cycloalkyl, heterocyclyl, heterocyclylalkyl, aryl, aralkyl, heteroaryl, heteroarylalkyl, OR⁶, CO₂R⁶, NR³(CO)R⁶, or (CO)NR³R⁶ wherein R³ and R⁶, taken together with the N to which they are attached, may form a heterocyclyl or heteroaryl moiety;
each R³ is independently hydrogen, alkyl, aryl or aralkyl;
R⁴ is O, S or NR³;
each R⁵ is independently H or alkyl;
each R⁶ is independently H, alkyl, heteroalkyl, cycloalkyl, heterocyclyl, heterocyclylalkyl, aryl, aralkyl, heteroaryl, heteroarylalkyl, alkoxy or fluoroalkoxy.

In some embodiments, R¹ is aryl-aryl or aryl-heteroaryl; in some embodiments, R¹ is aryl-heteroaryl; Preferably, the aryl ring attached to the core is a phenyl ring, substituted at a meta position with the aryl (e.g., phenyl) or heteroaryl (e.g., pyridyl, pyrimidyl, pyrazine, pyridazine, oxazole, etc.) ring. In some embodiments, R¹ aryl, heteroaryl or cycloalkyl.

In certain embodiments, R¹ is:

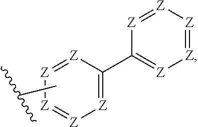

wherein
each Z is independently C—R′ or N; and
each R⁷ is independently H, alkyl, halo, heteroalkyl, cycloalkyl, heterocyclyl, heterocyclylalkyl, aryl, aralkyl, heteroaryl, heteroarylalkyl, alkoxy, fluoroalkoxy, OR⁶, NR⁶, CN, CO₂R⁶, NR³(CO)R⁶, or (CO)NR³R⁶ wherein R³ and R⁶, taken together with the N to which they are attached, may form a heterocyclyl, or heteroaryl moiety;
preferably:

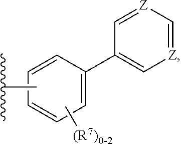

such as:

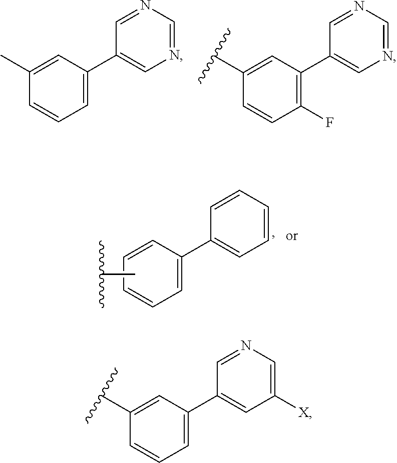

wherein X is F, Cl, Br, or I, preferably F.

In certain such embodiments, Z is selected in each case so that neither ring has more than four N, preferably no more than three N, even more preferably no more than two N (e.g., a pyrimidine ring). In preferred embodiments, the ring bound to the core is a substituted or unsubstituted phenyl ring, preferably attached to the core so that the attachment to the core and to the outer ring are in a 1,3 (meta) relationship on the phenyl ring. In preferred embodiments, the outer ring is a pyrimidine ring, most preferably a pyrimid-5-yl ring. In some embodiments, four Z are N; in some embodiments, three Z are N; in some embodiments, two Z are N.

In some embodiments, R¹ is:

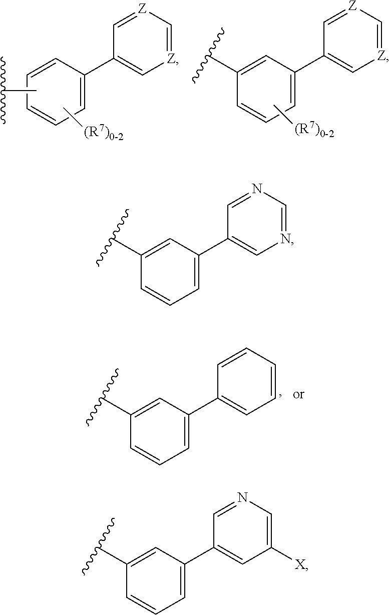

wherein X is F, Cl, Br, or I. In some embodiments, X is F.

In some embodiments, R¹ is:

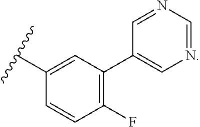 [SMILES: *c1ccc(F)c(-c2cncnc2)c1]

In some embodiments, R² is aryl or heteroaryl; in some embodiments R² is aryl; in some embodiments, R² is substituted phenyl; in some embodiments, R² is:

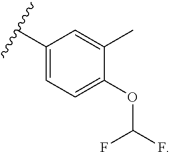 [SMILES: *c1ccc(OC(F)F)c(C)c1]

In some embodiments, R³ is alkyl; in some embodiments, R³ is methyl.

In some embodiments, R⁴ is O.

In some embodiments, R⁵ is H.

In some embodiments, the compound of formula (I) is of the structure

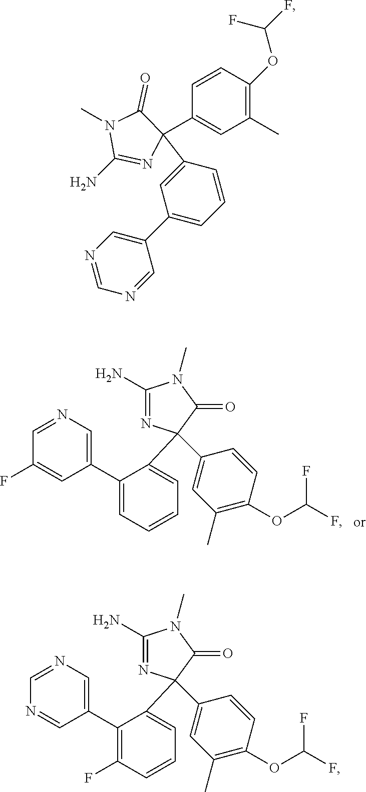 [SMILES: Cc1cc(C2(c3cccc(-c4cncnc4)c3)N=C(N)N(C)C2=O)ccc1OC(F)F ; Cc1cc(C2(c3ccccc3-c3cncc(F)c3)N=C(N)N(C)C2=O)ccc1OC(F)F ; Cc1cc(C2(c3cccc(F)c3-c3cncnc3)N=C(N)N(C)C2=O)ccc1OC(F)F]

or a pharmaceutically acceptable salt thereof.

In certain embodiments, the present disclosure provides a compound of formula I selected from the list consisting of:

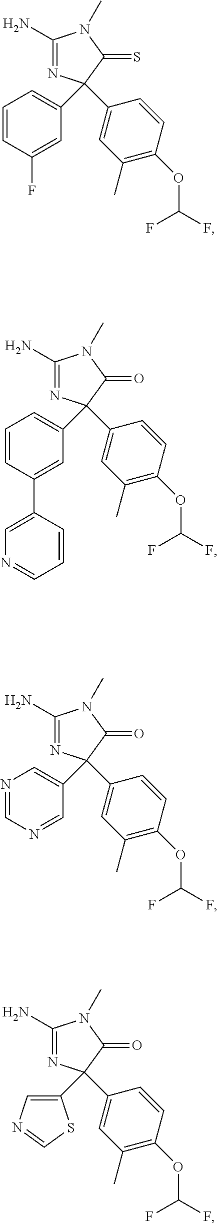 [SMILES: Cc1cc(C2(c3cccc(F)c3)N=C(N)N(C)C2=S)ccc1OC(F)F ; Cc1cc(C2(c3cccc(-c4cccnc4)c3)N=C(N)N(C)C2=O)ccc1OC(F)F ; Cc1cc(C2(c3cncnc3)N=C(N)N(C)C2=O)ccc1OC(F)F ; Cc1cc(C2(c3cncs3)N=C(N)N(C)C2=O)ccc1OC(F)F]

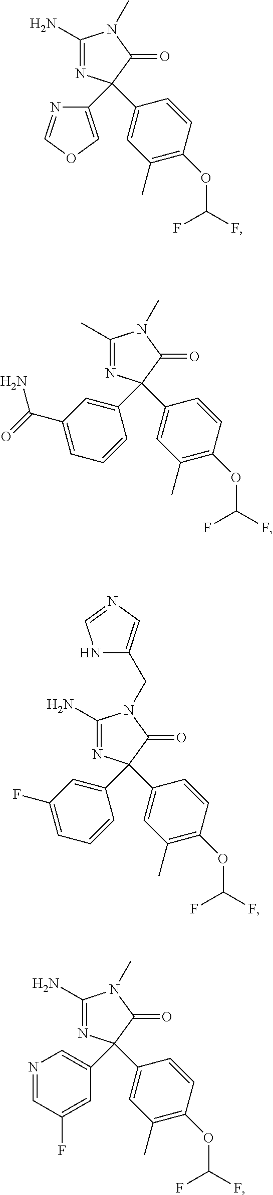 [SMILES: Cc1cc(C2(c3cocn3)N=C(N)N(C)C2=O)ccc1OC(F)F ; CC1=NC(c2cccc(C(N)=O)c2)(c2ccc(OC(F)F)c(C)c2)C(=O)N1C ; Cc1cc(C2(c3cccc(F)c3)N=C(N)N(Cc3cnc[nH]3)C2=O)ccc1OC(F)F ; Cc1cc(C2(c3cncc(F)c3)N=C(N)N(C)C2=O)ccc1OC(F)F]

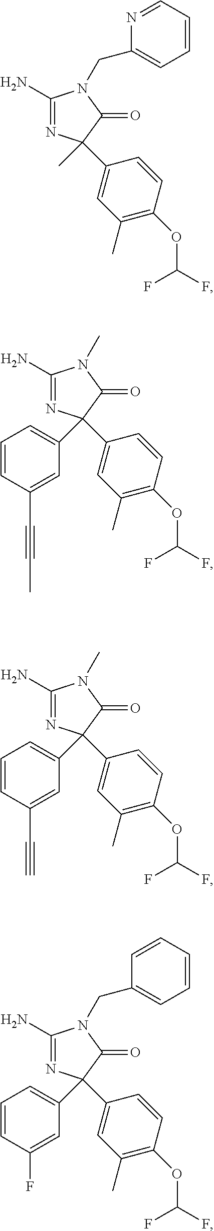 [SMILES: Cc1cc(C2(C)N=C(N)N(Cc3ccccn3)C2=O)ccc1OC(F)F ; CC#Cc1cccc(C2(c3ccc(OC(F)F)c(C)c3)N=C(N)N(C)C2=O)c1 ; C#Cc1cccc(C2(c3ccc(OC(F)F)c(C)c3)N=C(N)N(C)C2=O)c1 ; Cc1cc(C2(c3cccc(F)c3)N=C(N)N(Cc3ccccc3)C2=O)ccc1OC(F)F]

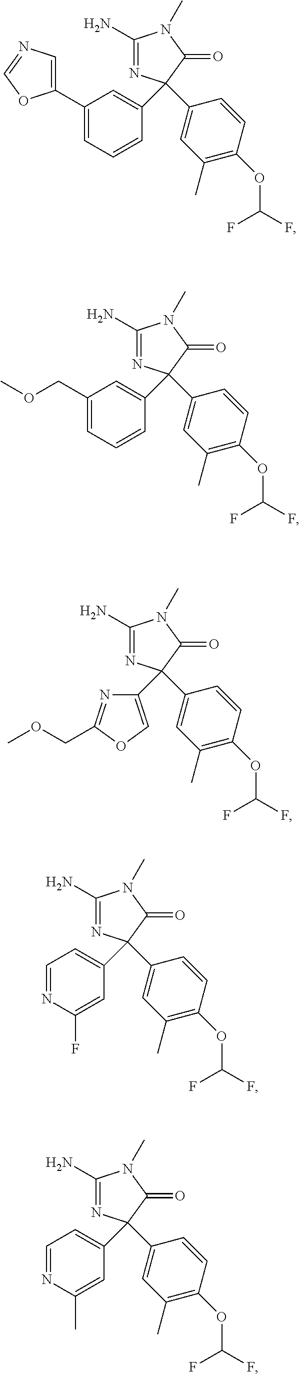 [SMILES: Cc1cc(C2(c3cccc(-c4cnco4)c3)N=C(N)N(C)C2=O)ccc1OC(F)F ; COCc1cccc(C2(c3ccc(OC(F)F)c(C)c3)N=C(N)N(C)C2=O)c1 ; COCc1nc(C2(c3ccc(OC(F)F)c(C)c3)N=C(N)N(C)C2=O)co1 ; Cc1cc(C2(c3ccnc(F)c3)N=C(N)N(C)C2=O)ccc1OC(F)F ; Cc1cc(C2(c3ccc(OC(F)F)c(C)c3)N=C(N)N(C)C2=O)ccn1]

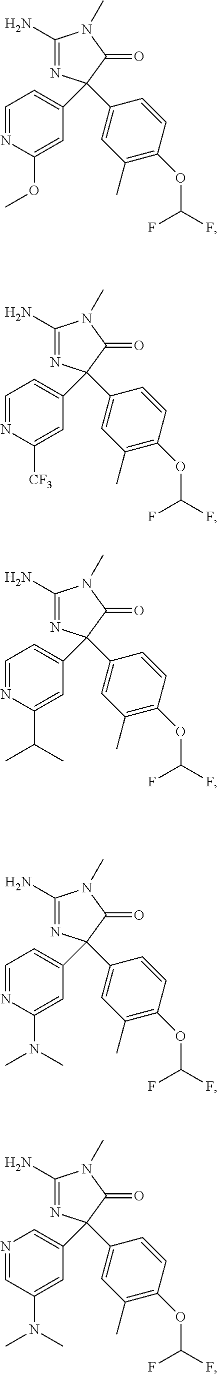 [SMILES: COc1cc(C2(c3ccc(OC(F)F)c(C)c3)N=C(N)N(C)C2=O)ccn1 ; Cc1cc(C2(c3ccnc(C(F)(F)F)c3)N=C(N)N(C)C2=O)ccc1OC(F)F ; Cc1cc(C2(c3ccnc(C(C)C)c3)N=C(N)N(C)C2=O)ccc1OC(F)F ; Cc1cc(C2(c3ccnc(N(C)C)c3)N=C(N)N(C)C2=O)ccc1OC(F)F ; Cc1cc(C2(c3cncc(N(C)C)c3)N=C(N)N(C)C2=O)ccc1OC(F)F]

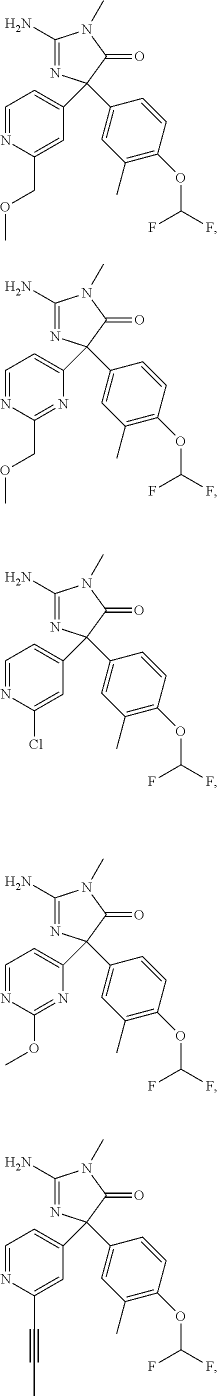 [SMILES: COCc1cc(C2(c3ccc(OC(F)F)c(C)c3)N=C(N)N(C)C2=O)ccn1 ; COCc1nccc(C2(c3ccc(OC(F)F)c(C)c3)N=C(N)N(C)C2=O)n1 ; Cc1cc(C2(c3ccnc(Cl)c3)N=C(N)N(C)C2=O)ccc1OC(F)F ; COc1nccc(C2(c3ccc(OC(F)F)c(C)c3)N=C(N)N(C)C2=O)n1 ; CC#Cc1cc(C2(c3ccc(OC(F)F)c(C)c3)N=C(N)N(C)C2=O)ccn1]

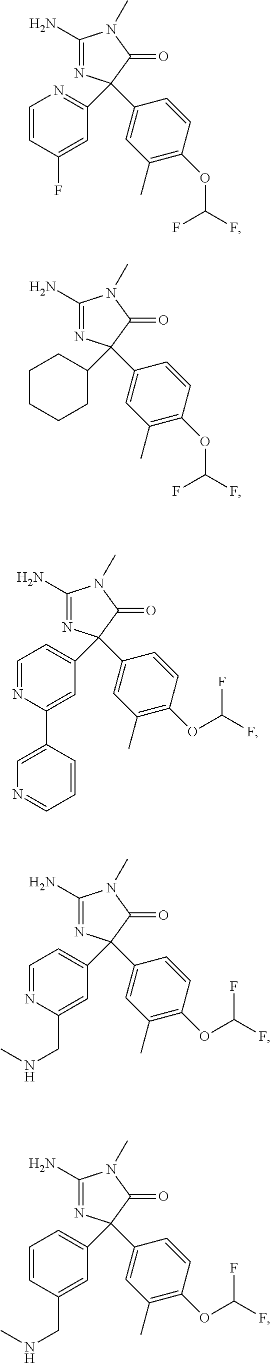 [SMILES: Cc1cc(C2(c3cc(F)ccn3)N=C(N)N(C)C2=O)ccc1OC(F)F ; Cc1cc(C2(C3CCCCC3)N=C(N)N(C)C2=O)ccc1OC(F)F ; Cc1cc(C2(c3ccnc(-c4cccnc4)c3)N=C(N)N(C)C2=O)ccc1OC(F)F ; CNCc1cc(C2(c3ccc(OC(F)F)c(C)c3)N=C(N)N(C)C2=O)ccn1 ; CNCc1cccc(C2(c3ccc(OC(F)F)c(C)c3)N=C(N)N(C)C2=O)c1]

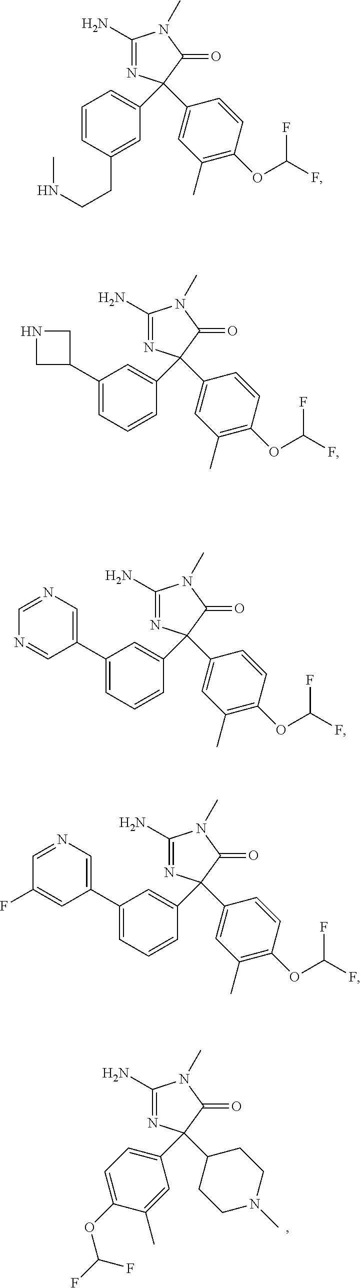 [SMILES: CNCCc1cccc(C2(c3ccc(OC(F)F)c(C)c3)N=C(N)N(C)C2=O)c1 ; Cc1cc(C2(c3cccc(C4CNC4)c3)N=C(N)N(C)C2=O)ccc1OC(F)F ; Cc1cc(C2(c3cccc(-c4cncnc4)c3)N=C(N)N(C)C2=O)ccc1OC(F)F ; Cc1cc(C2(c3cccc(-c4cncc(F)c4)c3)N=C(N)N(C)C2=O)ccc1OC(F)F ; Cc1cc(C2(C3CCN(C)CC3)N=C(N)N(C)C2=O)ccc1OC(F)F]

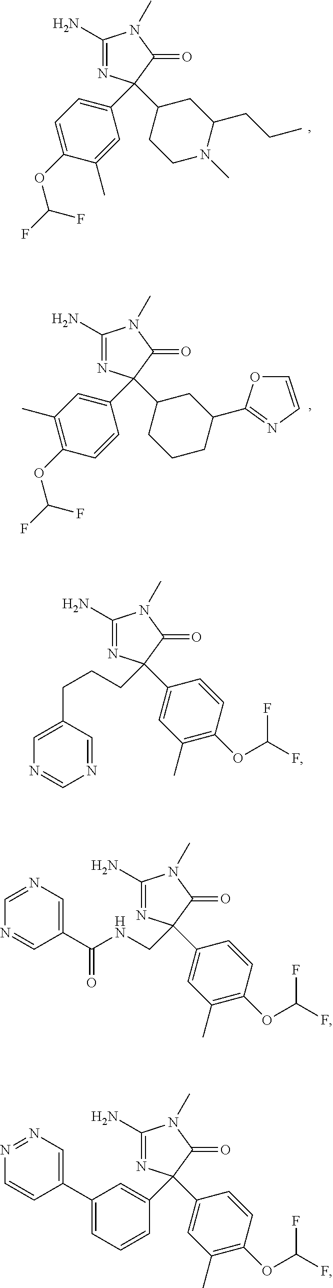

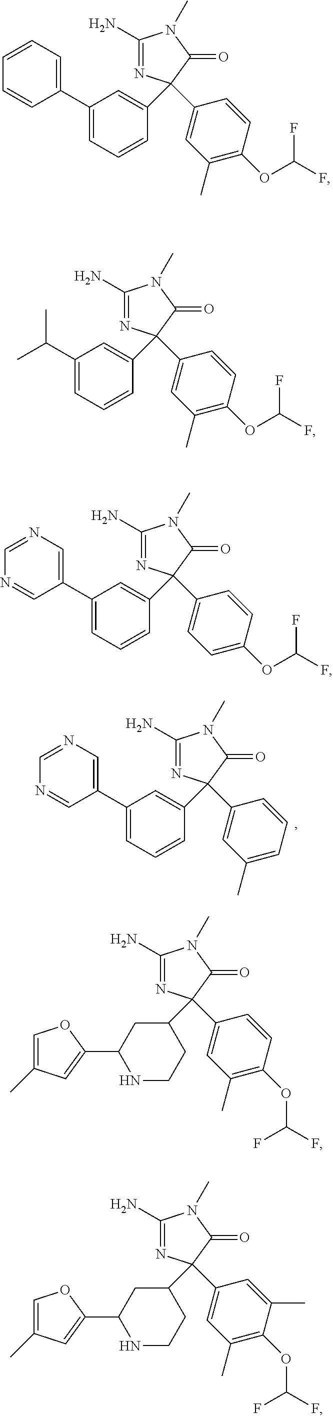

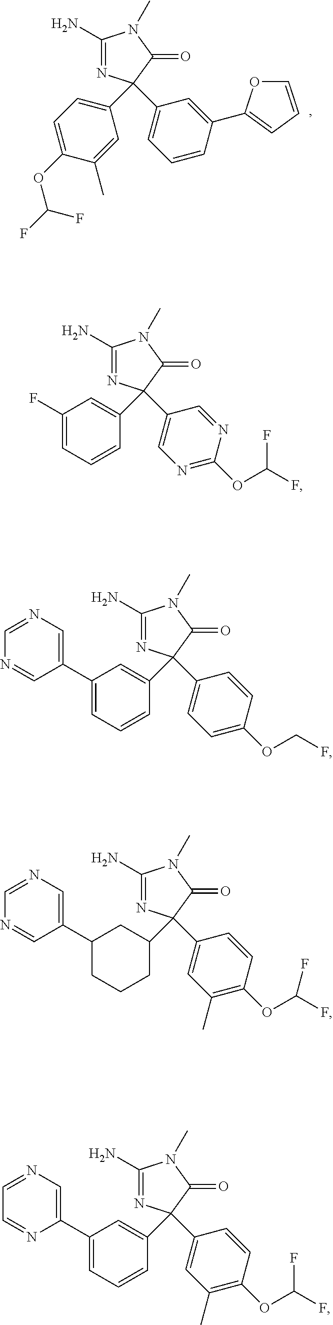 [SMILES: Cc1cc(C2(c3cccc(-c4ccco4)c3)N=C(N)N(C)C2=O)ccc1OC(F)F ; CN1C(=O)C(c2cnc(OC(F)F)nc2)(c2cccc(F)c2)N=C1N ; CN1C(=O)C(c2ccc(OCF)cc2)(c2cccc(-c3cncnc3)c2)N=C1N ; Cc1cc(C2(C3CCCC(c4cncnc4)C3)N=C(N)N(C)C2=O)ccc1OC(F)F ; Cc1cc(C2(c3cccc(-c4cnccn4)c3)N=C(N)N(C)C2=O)ccc1OC(F)F]

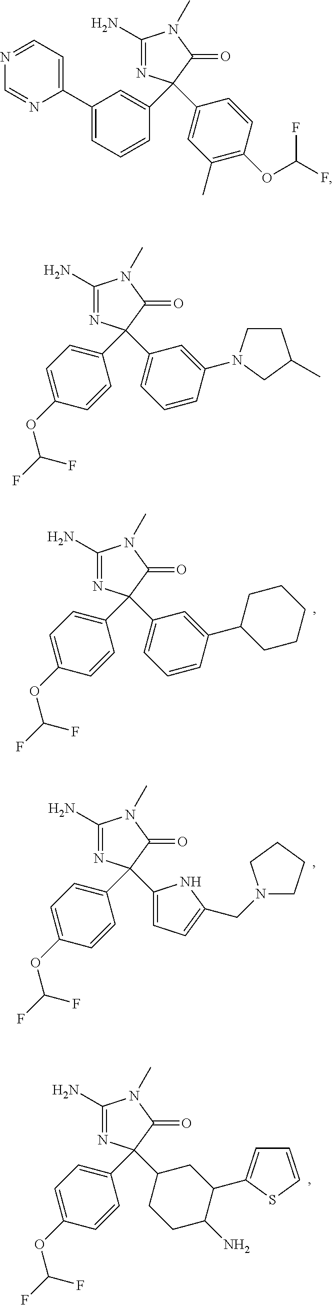 [SMILES: Cc1cc(C2(c3cccc(-c4ccncn4)c3)N=C(N)N(C)C2=O)ccc1OC(F)F ; CC1CCN(c2cccc(C3(c4ccc(OC(F)F)cc4)N=C(N)N(C)C3=O)c2)C1 ; CN1C(=O)C(c2ccc(OC(F)F)cc2)(c2cccc(C3CCCCC3)c2)N=C1N ; CN1C(=O)C(c2ccc(OC(F)F)cc2)(c2ccc(CN3CCCC3)[nH]2)N=C1N ; CN1C(=O)C(c2ccc(OC(F)F)cc2)(C2CCC(N)C(c3cccs3)C2)N=C1N]

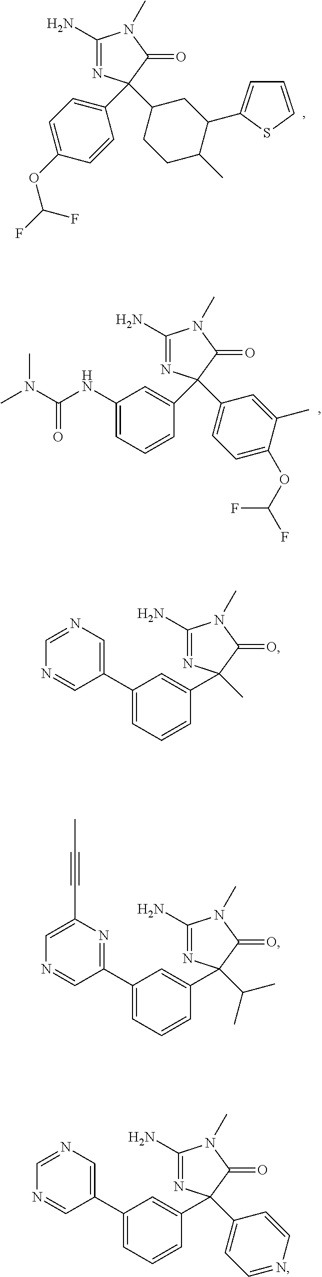

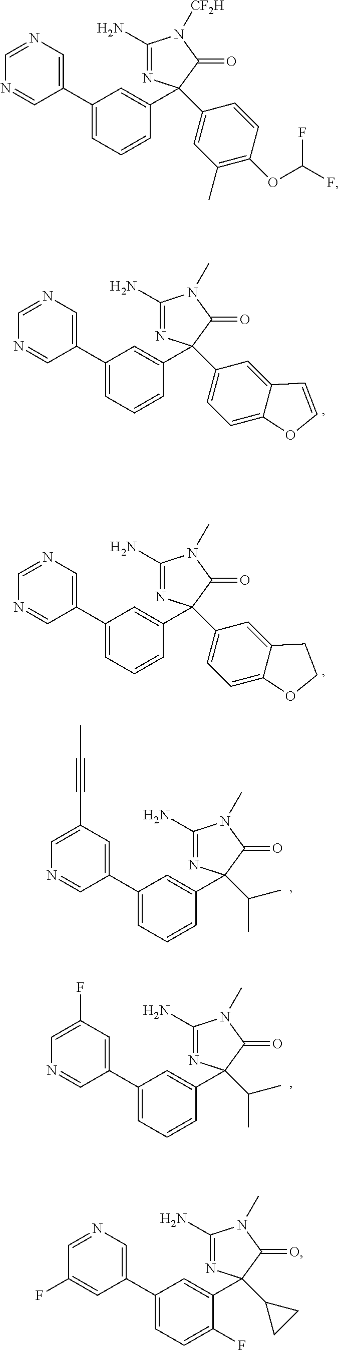 [SMILES: Cc1cc(C2(c3cccc(-c4cncnc4)c3)N=C(N)N(C(F)F)C2=O)ccc1OC(F)F ; CN1C(=O)C(c2cccc(-c3cncnc3)c2)(c2ccc3occc3c2)N=C1N ; CN1C(=O)C(c2cccc(-c3cncnc3)c2)(c2ccc3c(c2)CCO3)N=C1N ; CC#Cc1cncc(-c2cccc(C3(C(C)C)N=C(N)N(C)C3=O)c2)c1 ; CC(C)C1(c2cccc(-c3cncc(F)c3)c2)N=C(N)N(C)C1=O ; CN1C(=O)C(c2cc(-c3cncc(F)c3)ccc2F)(C2CC2)N=C1N]

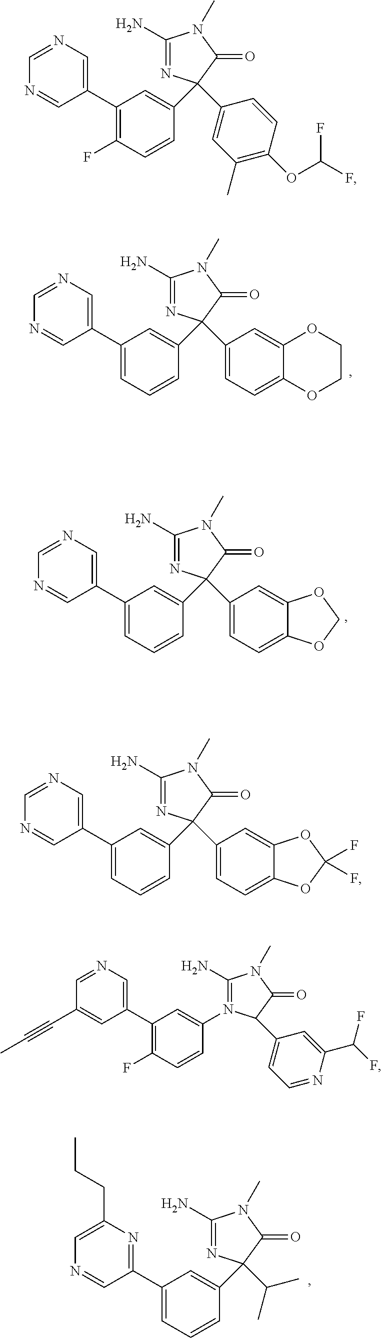

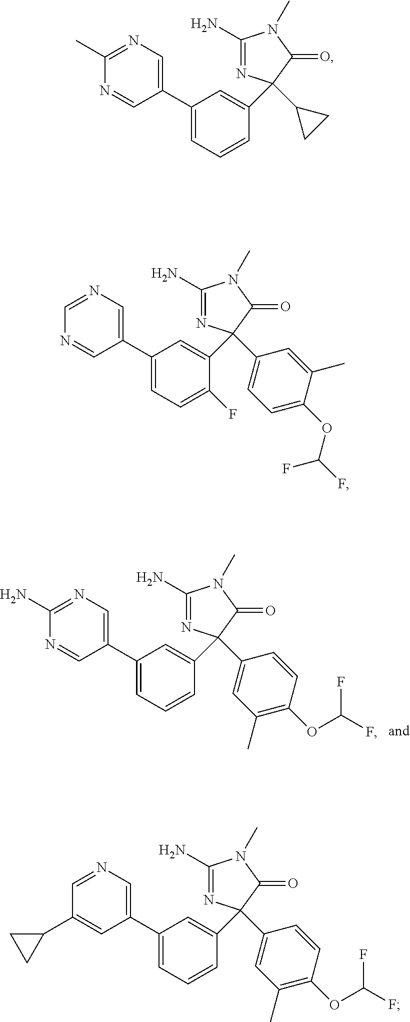 [SMILES: Cc1ncc(-c2cccc(C3(C4CC4)N=C(N)N(C)C3=O)c2)cn1 ; Cc1cc(C2(c3cc(-c4cncnc4)ccc3F)N=C(N)N(C)C2=O)ccc1OC(F)F ; Cc1cc(C2(c3cccc(-c4cnc(N)nc4)c3)N=C(N)N(C)C2=O)ccc1OC(F)F ; Cc1cc(C2(c3cccc(-c4cncc(C5CC5)c4)c3)N=C(N)N(C)C2=O)ccc1OC(F)F]

or a pharmaceutically acceptable salt thereof.

In another aspect, the present disclosure provides a pharmaceutical composition comprising a compound of formula I and a pharmaceutically acceptable excipient.

In another aspect, the present disclosure provides a method of treating Alzheimer's disease, comprising administering to a subject a compound of formula I.

In another aspect, the present disclosure provides method of treating Alzheimer's disease, comprising administering to a subject a pharmaceutical composition comprising a compound of formula I.

### Pharmaceutical Compositions

The compositions and methods of the present invention may be utilized to treat an individual in need thereof. In certain embodiments, the individual is a mammal such as a human, or a non-human mammal. When administered to a mammal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “acetal” is art-recognized and may be represented by the general formula

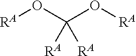

wherein each R^{A} independently represents hydrogen or a hydrocarbyl, such as alkyl, or any occurrence of R^{A} taken together with another and the intervening atom(s) complete a carbocycle or heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyl S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

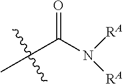

wherein each R^{A} independently represent a hydrogen, a hydrocarbyl, a herterocycle or a heteroaryl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

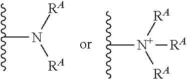

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “boron” as used herein with respect to a substituent on an organic compound, is art-recognized and refers to a group —B(R^{A})₂, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “boronic ester” or “boronate ester” as used herein is art-recognized and refers to a group —B(OR^{A})₂, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “carbamate” is art-recognized and refers to a group

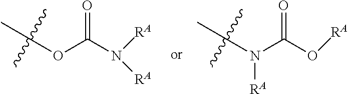

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “diazo”, as used herein, refers to a group represented by the formula ═N═N.

The term “disulfide” is art-recognized and refers to a group —S—S—R^{A}, wherein R^{A} represents a hydrocarbyl group.

The term “enol ester”, as used herein, refers to a group —C(O)O—C(R^{A})═C(R^{A})₂ wherein R^{A} represents a hydrocarbyl group.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “orthoester” as used herein is art-recognized and refers to a group —C(OR^{A})₃, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “phosphoester”, as used herein, refers to a group —P(O₂)OH. The term “phosphodiester”, as used herein, refers to a group —P(O₂)OR^{A} wherein R^{A} represents a hydrocarbyl group.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “selenide”, as used herein, is equivalent to an ether, wherein the oxygen is replaced with a selenium.

The term “selenoxide” is art-recognized and refers to the group —Se(O)—R^{A}, wherein R^{A} represents a hydrocarbyl.

The term “siloxane” is art-recognized and refers to a group with an Si—O—Si linkage, such as the group —Si(R^{A})₂—O—Si—(R^{A})₃, wherein each R^{A} independently represents hydrogen or hydrocarbyl, such as alkyl, or both R^{A} taken together with the intervening atom(s) complete a heterocycle having from 4 to 8 atoms in the ring structure.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

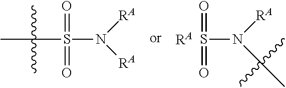

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

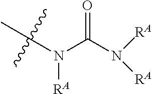

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

The invention now being generally described, it will be more readily understood by reference to the following examples which are included merely for purposes of illustration of certain aspects and embodiments of the present invention, and are not intended to limit the invention.

### EXAMPLES

### Example 1

### Synthesis of 2-amino-5-(4-(difluoromethoxy)-3-methylphenyl)-5-(4-fluoro-3-(pyrimidin-5-yl)phenyl)-3-methyl-3,5-dihydro-4H-imidazol-4-one (FAH-127)

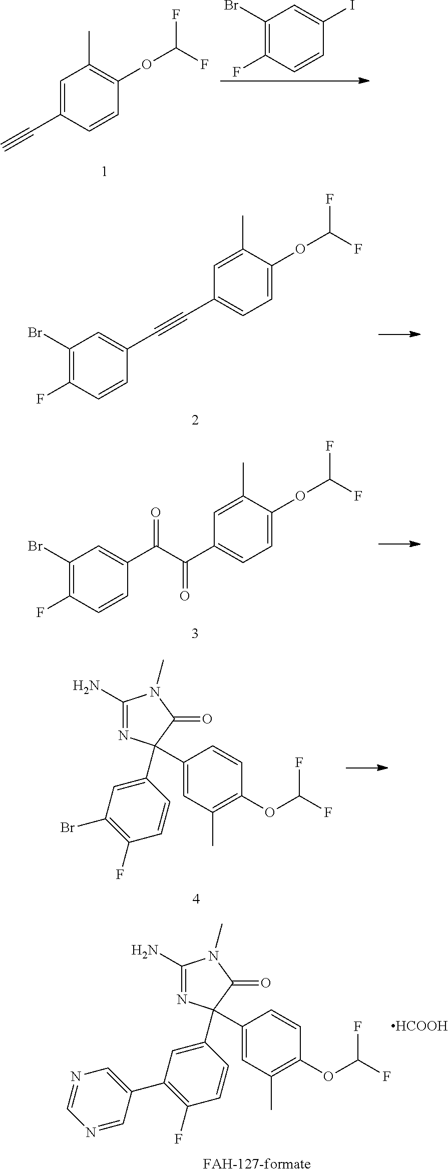

### Preparation of 2-bromo-4-((4-(difluoromethoxy)-3-methylphenyl)ethynyl)-1-fluorobenzene (2)

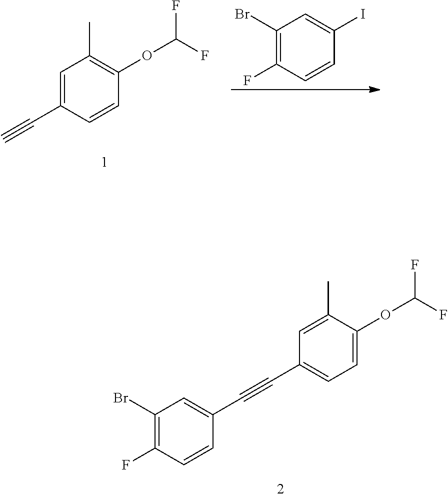 [SMILES: C#Cc1ccc(OC(F)F)c(C)c1 ; Fc1ccc(I)cc1Br ; Cc1cc(C#Cc2ccc(F)c(Br)c2)ccc1OC(F)F]

A solution of diisopropylethyamine (5 mL) and DMF (7 mL) was sparged with argon for about 15 minutes. 1-(difluoromethoxy)-4-ethynyl-2-methylbenzene (1) (550 mg, 3.02 mmol) and 2-bromo-1-fluoro-4-iodobenzene (1.36 g, 4.53 mmol) were added to the reaction mixture and argon gas was bubbled for another 5 minutes. Copper(I)iodide (57.5 mg, 0.302 mmol) and PdCl₂(PPh₃)₂ (106 mg, 151 μmol) were added to the reaction mixture and it was stirred at 60° C. overnight. The mixture was diluted with ether and washed with water. The mixture was filtered over Celite and the organic layer was separated. The aqueous layer was back extracted with ether. The combined organic layers were washed with saturated ammonium chloride solution, dried using sodium sulfate, filtered and the solvent was evaporated under reduced pressure. The crude mixture was partitioned with hexane and water. Some undesired solids were removed by filtration. The organic layer was separated and dried using sodium sulfate. The filtrate was evaporated under reduced pressure. The crude mixture was purified by flash chromatography using hexanes as the eluent 2-bromo-4-((4-(difluoromethoxy)-3-methylphenyl)ethynyl)-1-fluorobenzene (2) (1.0 g, 93.3% yield). ¹H NMR (400 MHz, DCM-d2) δ=7.79 (dd, J=2.0, 6.6 Hz, 1H), 7.50 (ddd, J=2.1, 4.7, 8.5 Hz, 1H), 7.46 (d, J=1.0 Hz, 1H), 7.40 (dd, J=2.0, 8.3 Hz, 1H), 7.17 (t, J=8.6 Hz, 1H), 7.10 (d, J=8.6 Hz, 1H), 6.82-6.41 (m, 1H), 2.33 (s, 3H).

### Preparation of 1-(3-bromo-4-fluorophenyl)-2-(4-(difluoromethoxy)-3-methylphenyl)ethane-1,2-dione (3)

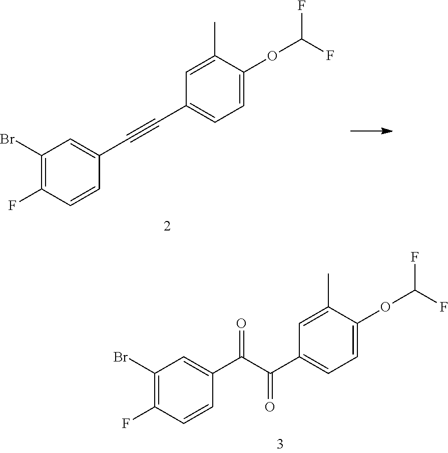 [SMILES: Cc1cc(C#Cc2ccc(F)c(Br)c2)ccc1OC(F)F ; Cc1cc(C(=O)C(=O)c2ccc(F)c(Br)c2)ccc1OC(F)F]

Dissolve 2-bromo-4-((4-(difluoromethoxy)-3-methylphenyl)ethynyl)-1-fluorobenzene (2) (1.0 g, 2.82 mmol) in DCM (40 mL) and AcOH (2 mL). Tetrabutylammonium bromide (1.36 g, 4.22 mmol) and potassium permanganate (1.30 g, 8.22 mmol) were added to the reaction mixture, which was then stirred at room temperature for 2 hours. The mixture was diluted with DCM and was quenched with 5% sodium metabisulphite. The organic layer was separated and dried using sodium sulfate. The crude mixture was purified using flash chromatography using hexane and ethyl acetate as gradients 1-(3-bromo-4-fluorophenyl)-2-(4-(difluoromethoxy)-3-methylphenyl)ethane-1,2-dione (3) (910 mg, 84%). ¹H NMR (400 MHz, DCM-d2) δ=8.23 (dd, J=2.0, 6.6 Hz, 1H), 7.94 (ddd, J=2.1, 4.7, 8.6 Hz, 1H), 7.87 (d, J=1.3 Hz, 1H), 7.82 (dd, J=2.1, 8.7 Hz, 1H), 7.29 (t, J=8.3 Hz, 1H), 7.19 (d, J=8.6 Hz, 1H), 6.89 6.47 (m, 1H), 2.34 (s, 3H).

### Preparation of 2-amino-5-(3-bromo-4-fluorophenyl)-5-(4-(difluoromethoxy)-3-methylphenyl)-3-methyl-3,5-dihydro-4H-imidazol-4-one (4)

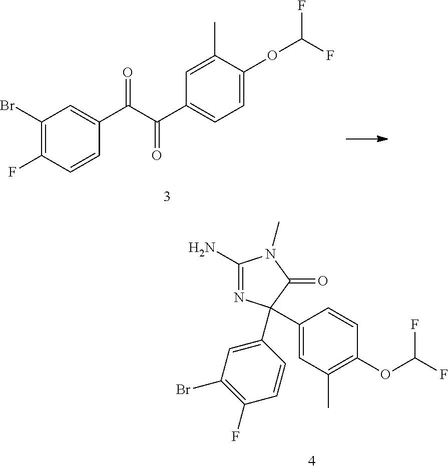 [SMILES: Cc1cc(C(=O)C(=O)c2ccc(F)c(Br)c2)ccc1OC(F)F ; Cc1cc(C2(c3ccc(F)c(Br)c3)N=C(N)N(C)C2=O)ccc1OC(F)F]

To a solution of 1-(3-bromo-4-fluorophenyl)-2-(4-(difluoromethoxy)-3-methylphenyl)ethane-1,2-dione (3) (910 mg, 2.35 mmol) in isopropanol (5 mL), sodium carbonate (436 mg, 4.11 mmol) and 1-methyl guanidine hydrochloride (386 mg, 3.53 mmol) were added and the mixture was stirred at 80° C. for 2 hours. The reaction mixture was cooled to room temperature. The filtrate was concentrated and loaded directly onto a silica gel column and purified by flash chromatography using a gradient of MeOH:DCM to obtain 2-amino-5-(3-bromo-4-fluorophenyl)-5-(4-(difluoromethoxy)-3-methylphenyl)-3-methyl-3,5-dihydro-4H-imidazol-4-one (4) (610 mg, 59%). ¹H NMR (400 MHz, DCM-d2) δ=7.73 (dd, J=1.5, 6.6 Hz, 1H), 7.44 (ddd, J=1.8, 4.4, 8.5 Hz, 1H), 7.31 (s, 1H), 7.26 (br d, J=8.6 Hz, 1H), 7.07 (t, J=8.6 Hz, 1H), 7.00 (d, J=8.3 Hz, 1H), 6.73-6.30 (m, 1H), 3.07 (s, 3H), 2.24 (s, 3H).

### Synthesis of 2-amino-5-(4-(difluoromethoxy)-3-methylphenyl)-5-(4-fluoro-3-(pyrimidin-5-yl)phenyl)-3-methyl-3,5-dihydro-4H-imidazol-4-one formate (FAH-127-Formate)

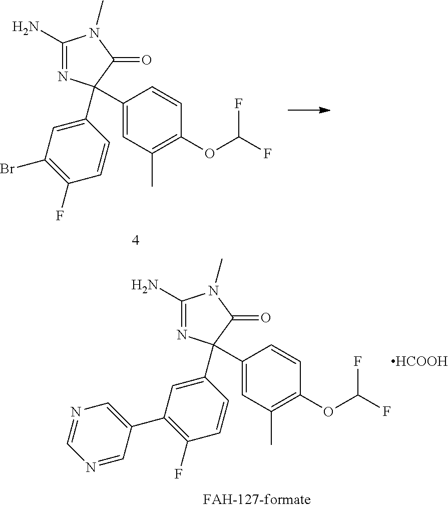 [SMILES: Cc1cc(C2(c3ccc(F)c(Br)c3)N=C(N)N(C)C2=O)ccc1OC(F)F ; Cc1cc(C2(c3ccc(F)c(-c4cncnc4)c3)N=C(N)N(C)C2=O)ccc1OC(F)F ; "·HCOOH"]

Dissolved 2-amino-5-(3-bromo-4-fluorophenyl)-5-(4-(difluoromethoxy)-3-methylphenyl)-3-methyl-3,5-dihydro-4H-imidazol-4-one (4) (610 mg, 1.38 mmol) in dioxane (3 mL). Pyrimidine-5-boronic acid (171 mg, 1.38 mmol) and 2 M potassium carbonate solution (2.07 mL) were added. Argon was bubbled through the mixture. Xphos Pd Gen2 (109 mg, 138 μmol) was added and the mixture was heated at 100° C. overnight. The reaction mixture was separated and the aqueous layer was extracted with chloroform. The combined organic layers were washed with brine, dried using sodium sulfate, and concentrated under reduced pressure. The crude mixture was purified using flash chromatography using a gradient of DCM and 1:9 MeOH:DCM. The obtained solid was dissolved in 1:4 acetonitrile:water and 3 eq of formic acid were added and the resultant solution was lyophilized to yield a powder 2-amino-5-(4-(difluoromethoxy)-3-methylphenyl)-5-(4-fluoro-3-(pyrimidin-5-yl)phenyl)-3-methyl-3,5-dihydro-4H-imidazol-4-one (FAH-127-Formate) (322 mg, 48%). ¹H NMR (300 MHz, MeOH-d4) δ=9.16 (s, 1H), 8.96 (bs, 2H), 7.62 (m, 1H), 7.55 (m, 1H), 7.29 (m, 3H), 7.12 (m, 1H), 7.06 to 6.56 (m, 1H), 3.20 (s, 3H), 2.25 (s, 3H).

### Example 2

### Synthesis of 2-amino-5-(4-(difluoromethoxy)-3-methylphenyl)-3-methyl-5-(3-(pyridin-3-yl)phenyl)-3,5-dihydro-4Himidazol-4-one (12)

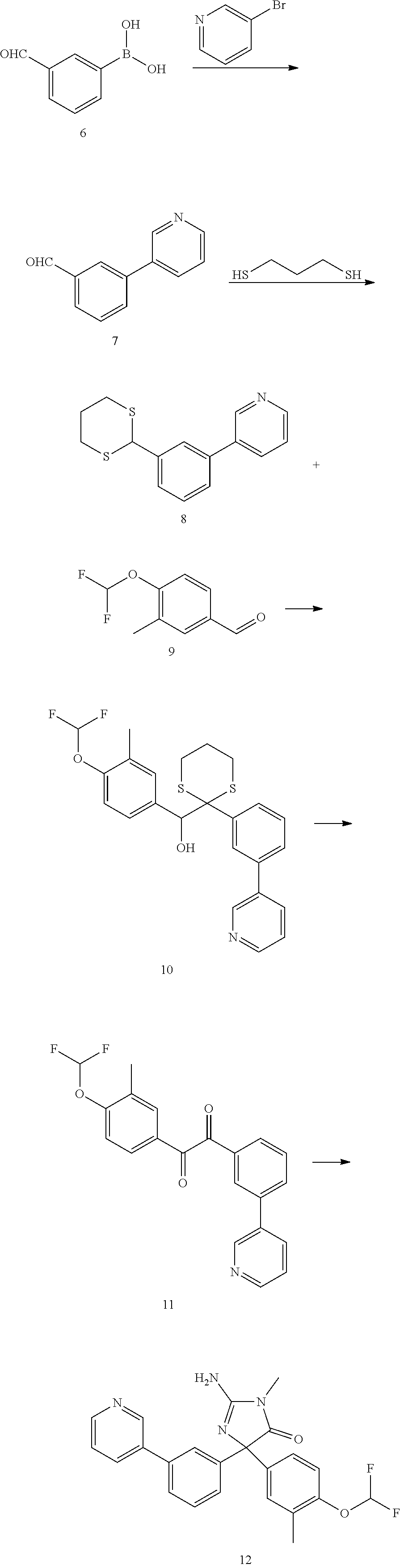

### Preparation of 3-(pyridin-3-yl)benzaldehyde (7)

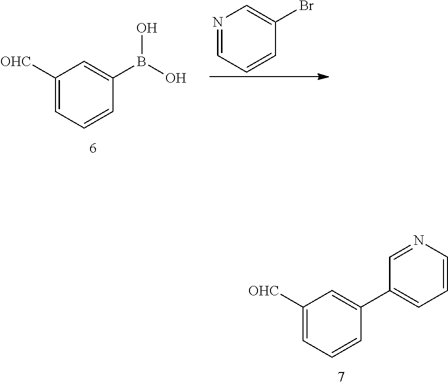 [SMILES: O=Cc1cccc(B(O)O)c1 ; Brc1cccnc1 ; O=Cc1cccc(-c2cccnc2)c1]

3-Bromopyridine (1.46 g, 924.1 mmol, 1.0 eq), 3-formylphenylboronic acid (6) (1.524 g, 1016.5 mmol, 1.10 eq), and potassium carbonate (3.831 g, 27.7 mmol, 3.0 eq) were dissolved in DMF (35 mL) and water (10 mL). The mixture was degassed and backfilled with nitrogen (the process was repeated twice). Following degassing, PdCl₂(PPh₃)₂ (0.195 g, 0.3 mmol) was added and the mixture was heated at 100° C. for 2 h. The reaction was then concentrated and the residue partitioned between ethyl acetate (180 mL) and water (100 mL). The organic layer was washed with additional water (2×100 mL), brine, dried over sodium sulfate, and concentrated. The resulting oil was dissolved in DCM and chromatographed over silica gel (50-100% EA/Hex gradient) to 3-(pyridin-3-yl)benzaldehyde (7) as an oil (1.16 g, 68.71%). ¹H NMR (300 MHz, CDCl₃), δ 10.1 (s, 1H), 8.89 (s, 1H), 8.65 (d, J=4.8 Hz, 1H), 8.10 (s, 1H), 7.93 (d, J=7.8 Hz, 2H), 7.86 (d, J=6.9 Hz, 1H), 7.67 (t, J=7.6 Hz, 1H), 7.44-7.40 (m, 1H).

### Preparation of 3-(3-1,3-dithian-2-yl)phenyl)pyridine (8)

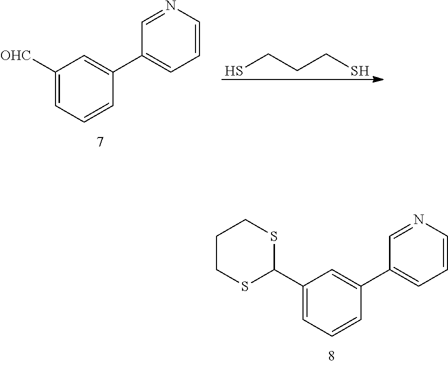

BF₃OEt₂ (0.67 mL, 5.4 mmol, 0.86 eq) was added dropwise to a solution of 1,3-propanedithiol (0.64 mL, 6.3 mmol, 1.0 eq) and 3-(pyridine-3-yl)benzaldehyde (7) (1.163 g, 6.3 mmol, 1.0 eq) in DCM (30 mL) at 0° C. The reaction was stirred at ambient temperature for 1 hour when TLC (30% EtOAc/hexane) indicated incomplete reaction. The reaction was allowed to stir at the same temperature overnight. TLC (30% EtOAc/hexane) indicated the reaction was not complete. 1,3-Propanedithiol (0.64 mL, 6.3 mmol, 1.0 eq) was added to the reaction mixture and then BF₃OEt₂ (0.67 mL,5.4 mmol, 0.86 eq) was added dropwise at 0° C. The ice bath was removed and the reaction was stirred at room temperature for 1 hr. TLC (30% EtOAc/hexane) indicated the reaction was complete. The reaction mixture was then diluted with DCM (50 mL) and filtered through Celite pad. Celite pad was washed with additional DCM (3×50 mL). The combined filtrates were washed with saturated NaHCO₃ (3×100 mL), 10% KOH solution (100 mL), water (80 mL) and brine (100 mL) and finally dried over anhydrous sodium sulfate. The organic extract was filtered and evaporated. The resulting residue was dissolved in DCM and chromatographed over silica gel (30-100% ethyl acetate:hexane gradient) to afford 3-(3-(1,3-dithian-2-yl)phenyl)pyridine (8) (1.234 g, 4.5 mmol, 71.43%) as a white solid (1.23 g, 71%). ¹H NMR (300 MHz, CDCl₃), δ 8.88 (s, 1H), 8.63 (d, J=5.1 Hz, 1H), 8.08 (d, J=6.9 Hz, 1H), 7.71 (s, 1H), 7.54-7.46 (m, 4H), 5.24 (s, 1H), 3.15-2.91 (m, 4H), 2.24-1.94 (m, 2H). (80 ml)

### Preparation of 4-(difluoromethoxy)-3-methylbenzaldehyde (9)

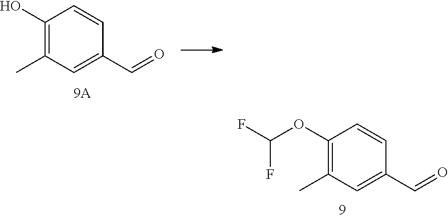

Potassium hydroxide (8.176 g, 146.0 mmol, 20.0 eq) was suspended in a mixture of acetonitrile (15 mL) and water (15 mL) and cooled to −20° C. 4-Hydroxy-3-methyl-benzaldehyde (9A) (1.000 g, 7.3 mmol, 1.0 eq) was dissolved in acetonitrile (10 mL) and was added dropwise, followed by bromodifluoromethyl diethylphosphonate (3.811 g, 14.6 mmol, 2.0 eq) over 15 minutes. Then the mixture was allowed to warm to room temperature over 1 h. The mixture was extracted with ethyl acetate (3×30 mL). Then it was washed with brine (2×30 mL) and finally dried over sodium sulfate. The combined organics were removed under reduced pressure. Resulting residue was dissolved in DCM and loaded to silica gel column (12 g, 0-20% EA/Hex, TLC 10% EA/Hex). The title compound was obtained as clear oil (9) (0.454 g, 33.4%). ¹H NMR (300 MHz, CDCl₃), δ9.95 (s, 1H), 7.77-7.71 (m, 2H), 7.26-7.19 (m, 1H), 6.62 (t, J=72.9 Hz, 1H), 2.36 (s, 3H).

### Preparation of (4-(difluoromethoxy)-3-methylphenyl)(2-(3-(pyridin-3-yl)phenyl)-1,3-dithian-2-yl)methanol (10)

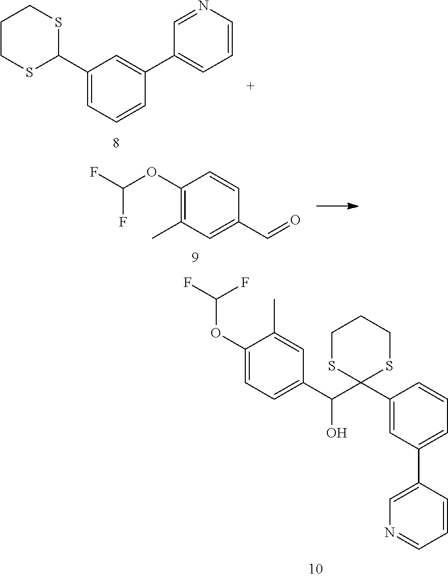 [SMILES: c1cncc(-c2cccc(C3SCCCS3)c2)c1 ; Cc1cc(C=O)ccc1OC(F)F ; Cc1cc(C(O)C2(c3cccc(-c4cccnc4)c3)SCCCS2)ccc1OC(F)F]

3-(3-(1,3-dithian-2-yl)phenyl)pyridine (8) (0.656 g, 2.4 mmol, 1.0 eq) was dissolved in dry THF (13.0 mL) and cooled to −20° C. nBuLi (2.5 M, 1.160 mL, 2.9 mmol) was added dropwise under nitrogen and the mixture was stirred for 30 min at −10° C. to afford a dark red solution. A solution of 4-(difluoromethoxy)-3-methylbenzaldehyde (9) (0.454 g, 2.4 mmol, 1.0 eq) in THF (13.0 mL) was added dropwise and the mixture was stirred at −20° C. for 15 min, then warmed to ambient temperature over 1 h and quenched with saturated ammonium chloride solution (1.5 mL) followed by dilution with EtOAc (10 mL). The organic phase was washed ammonium chloride solution (1.5 mL) followed by dilution with EtOAc (10 mL). The organic phase was washed with water (2×10 mL), brine (20 mL) and dried with anhydrous sodium sulfate. After filtration and concentration the crude product was purified by flash chromatograph to give (4-(difluoromethoxy)-3-methylphenyl)(2-(3-(pyridin-3-yl)phenyl)-1,3-dithian-2-yl)methanol (10) as a white solid (0.505 g, 50%). ¹H NMR (300 MHz, CDCl3), δ 8.75 (s, 1H), 8.61 (d, J=4.8 Hz, 1H), 7.92 (d, J=6.3 Hz, 1H), 7.83 (d, J=6.3 Hz, 1H), 7.74 (s, 1H), 7.55-7.47 (m, 3H), 6.85 (d, J=8.1 Hz, 1H), 6.72-6.21 (m, 3H), 4.99 (s, 1H), 2.81-2.60 (m, 4H), 2.09 (s, 3H), 1.98-1.95 (m, 2H).

### Preparation of 1-(4-(difluoromethoxy)-3-methylphenyl)-2-(3-(pyridin-3-yl)phenyl)ethane-1,2-dione (11)

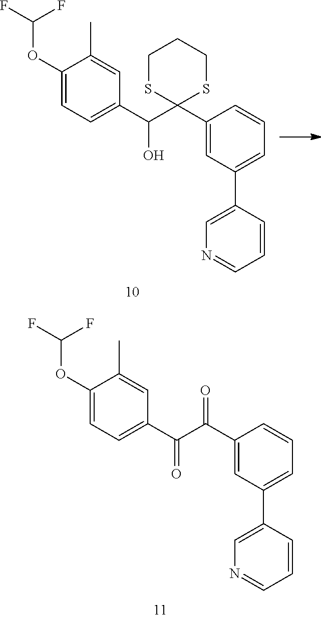 [SMILES: Cc1cc(C(O)C2(c3cccc(-c4cccnc4)c3)SCCCS2)ccc1OC(F)F ; Cc1cc(C(=O)C(=O)c2cccc(-c3cccnc3)c2)ccc1OC(F)F]

(4-(difluoromethoxy)-3-methylphenyl)(2-(3-(pyridin-3-yl)phenyl)-1,3-dithian-2-yl)methanol (10) (0.50 g, 1.1 mmol, 1.0 eq) was dissolved in dichloromethane (13.89 mL) and tert-butanol (2.94 mL, 30.8 mmol, 28.0 eq). Dess-Martin periodinane (1.17 g, 2.8 mmol, 2.5 eq) was added and the reaction was stirred overnight at room temperature. The mixture was extracted with ethyl acetate (30 mL). The organic phase was washed with sodium hydrogen carbonate, brine and dried over anhydrous sodium sulfate. The solvent was evaporated and crude product was purified by flash chromatograph to give 1-(4-(difluoromethoxy)-3-methylphenyl)-2-(3-(pyridin-3-yl)phenyl)ethane-1,2-dione (11) (0.1718 g, 45%) as a white solid. ¹H NMR (300 MHz, CDCl₃), δ 8.90 (s, 1H), 8.69 (s, 1H), 8.20 (s, 1H), 8.12-7.85 (m, 5H), 7.70-7.57 (m, 2H), 7.19 (d, J=8.7 Hz, 1H), 6.63 (t, J=72.6 Hz, 1Hz), 2.35 (s, 1H).

### Preparation of 2-amino-5-(4-(difluoromethoxy)-3-methylphenyl)-3-methyl-5-(3-(pyridin-3-yl)phenyl)-3,5-dihydro-4H-imidazol-4-one (12)

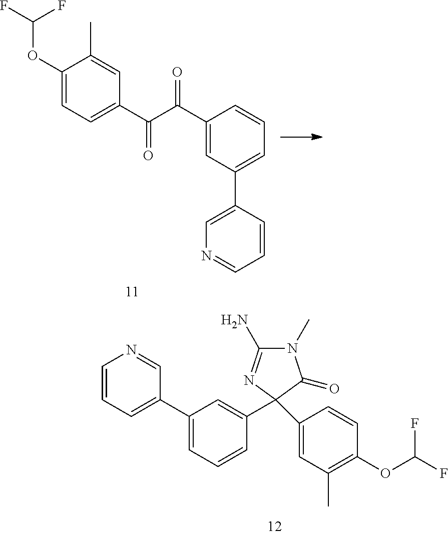 [SMILES: Cc1cc(C(=O)C(=O)c2cccc(-c3cccnc3)c2)ccc1OC(F)F ; Cc1cc(C2(c3cccc(-c4cccnc4)c3)N=C(N)N(C)C2=O)ccc1OC(F)F]

Sodium carbonate (0.170 g, 1.6mmol, 4.0 eq) in water (2.406 mL) was added to a mixture of 1-(4-(difluoromethoxy)-3-methylphenyl)-2-(3-(pyridin-3-yl)phenyl)ethane-1,2-dione (11)(0.135 g, 0.4 mmol, 1.0 eq), 1-methylguanidine hydrochloride (0.176 g, 1.6 mmol, 4.0 eq), dioxane (6.211 mL), and ethyl alcohol (8.062 mL). The reaction mixture was stirred at 85° C. for 5 hrs. Mixture was cooled to room temperature and volatiles were removed in vacuo, residue was dissolved in DCM (30 mL) and washed with water (2×2 0 mL), dried over anhydrous sodium sulfate, filtered and evaporated. Crude product was purified by flash chromatography to give 2-amino-5-(4-(difluoromethoxy)-3-methylphenyl)-3-methyl-5-(3-(pyridin-3-yl)phenyl)-3,5-dihydro-4Himidazol-4-one as a white solid (12) (67 mg, 40%). HPLC 99.5%, LCMS, [M+1]⁺423.1. ¹H NMR (300 MHz, DMSO-d6), δ 8.77 (brs, 1H), 8.57 (d, J=4.2 Hz, 1H), 7.94 (d, J=7.5 Hz, 1H), 7.75 (s, 1H), 7.60-7.36 (m, 6H), 7.11-6.87 (m, 2H), 6.73 (brs, 2H), 2.99 (s, 3H), 2.18 (s, 3H).

### Example 3

### Synthesis of 5-amino-3-(4-(difluoromethoxy)-3-methylphenyl)-1-methyl-3-(3-(pyrimidin-5-yl)phenyl)-1,3-dihydro-2H-pyrrol-2-one (FAH-65)

### Preparation of 5-(3-bromophenyl)pyrimidine (14)

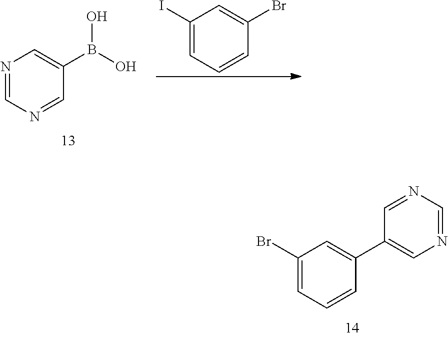 [SMILES: OB(O)c1cncnc1 ; Brc1cccc(I)c1 ; Brc1cccc(-c2cncnc2)c1]

Pyrimidine-5-boronic acid (13) (2.0 g, 16.1 mmol) was dissolved in mixture of water (8 mL), ethanol (16 mL) and toluene (16 mL) and 1-bromo-3-iodobenzene (4.57 g, 16.1 mmol) followed by potassium carbonate (4.46 g, 32.3 mmol, 2 eq) and Pd(dppf)Cl2 (1.18 g, 1.61 mmol, 0.1 eq) were added. Mixture was stirred at room temp for 1 min and temperature was increased slowly to 150° C. over 2 hrs. Mixture was allowed to cool to room temperature, and poured into mixture of 200 mL of ethyl acetate and 100 mL of water, shaken well, organic layer separated and washed with brine 2×, filtered through pad of Celite, dried with anh. sodium sulfate, filtered and concentrated. Product 5-(3-bromophenyl)pyrimidine (14) was purified by flash chromatography, yield 2.42 g, light-pink solid.

### Preparation of 5-(3-((4-(difluoromethoxy)-3-methylphenyl)ethynyl)phenyl)pyrimidine (16)

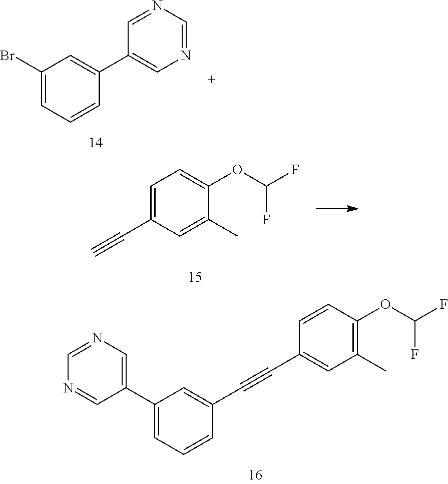 [SMILES: Brc1cccc(-c2cncnc2)c1 ; C#Cc1ccc(OC(F)F)c(C)c1 ; Cc1cc(C#Cc2cccc(-c3cncnc3)c2)ccc1OC(F)F]

5-(3-bromophenyl)pyrimidine (14) (1.20 g, 5.10 mmol) and 1-(difluoromethoxy)-4-ethynyl-2-methylbenzene (15) (1.39 g, 7.66 m mol, 1.5 e q) w ere dissolved in DMF (36 mL). To this stirred solution was added palladium dichlorobis(triphenylphosphine) (179 mg, 255 μmol, 50 meq), copper(1)iodide (97.2 mg, 510 μmol, 0.1 eq) and Hünig's base (1.76 ml, 10.2 mmol, 2 eq). Resulted mixture was heated at 65° C. under argon overnight. On cooling to room temperature mixture was diluted with ethyl acetate (200 mL) washed with water (3×), brine (1×), organic layer separated, dried over anh. sodium sulfate, filtered and concentrated. Product was purified by flash chromatography; yield 5-(3-((4-(difluoromethoxy)-3-methylphenyl)ethynyl)phenyl)pyrimidine (16) 1.503 g, 87.5%, brown liquid.

### Preparation of 1-(4-(difluoromethoxy)-3-methylphenyl)-2-(3-(pyrimidin-5-yl)phenyl)ethane-1,2-dione (17)

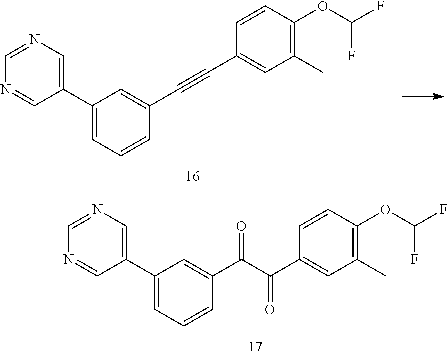 [SMILES: Cc1cc(C#Cc2cccc(-c3cncnc3)c2)ccc1OC(F)F ; Cc1cc(C(=O)C(=O)c2cccc(-c3cncnc3)c2)ccc1OC(F)F]

5-(3-((4-(difluoromethoxy)-3-methylphenyl)ethynyl)phenyl)pyrimidine (16) (730 mg, 2.17 mmol) was dissolved in 5% AcOH in DCM (60 mL). To this stirred solution was added tetrabutylammonium bromide (1.05 g, 3.26 mmol, 1.5 eq), followed by potassium permanganate (1.03 g, 6.51 mmol, 3 eq) and mixture was stirred for 1 hr. Reaction mixture was diluted with DCM (200 mL) and quenched with 5% sodium metabisulfite in water (200 mL). Biphasic mixture turned light yellow. The organic layer was separated, dried with anh. sodium sulfate and filtered through 1 in plug of silica gel. Silica gel was washed with EtOAc until all product was eluted. Crude product was purified by flash chromatography to give 98 mg of pure product 1-(4-(difluoromethoxy)-3-methylphenyl)-2-(3-(pyrimidin-5-yl)phenyl)ethane-1,2-dione (17).

### Preparation of 5-amino-3-(4-(difluoromethoxy)-3-methylphenyl)-1-methyl-3-(3-(pyrimidin-5-yl)phenyl)-1,3-dihydro-2H-pyrrol-2-one (FAH-65)

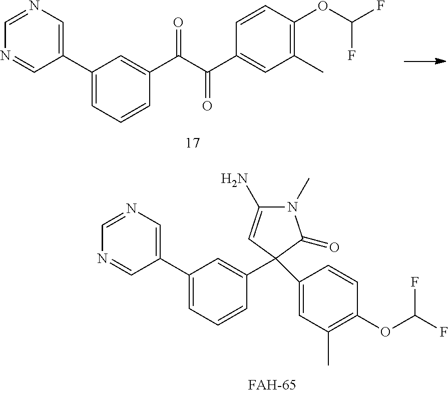 [SMILES: Cc1cc(C(=O)C(=O)c2cccc(-c3cncnc3)c2)ccc1OC(F)F ; Cc1cc(C2(c3cccc(-c4cncnc4)c3)C=C(N)N(C)C2=O)ccc1OC(F)F]

1-(4-(difluoromethoxy)-3-methylphenyl)-2-(3-(pyrimidin-5-yl)phenyl)ethane-1,2-dione (17) (98 mg, 266 μmol) was dissolved in isopropanol (2.0 mL and 1-methylguanidine hydrochloride (43.7 mmg, 399 μmol, 1.5 eq) was added, followed by anhydrous sodium carbonate (42.3 mg, 399 μmol, 1.5 eq). The reaction mixture was heated at 80° C. overnight. On cooling to room temperature mixture was diluted with chloroform (5 mL), washed with water and brine, dried with anhydrous sodium sulfate, filtered and concentrated. Product was purified by flash chromatography to give pure product 5-amino-3-(4-(difluoromethoxy)-3-methylphenyl)-1-methyl-3-(3-(pyrimidin-5-yl)phenyl)-1,3-dihydro-2H-pyrrol-2-one (FAH-65), 83 mg as off-white foam. LCMS (ESI) [M+H]⁺=424.4. ¹H NMR (600 MHz, MeOH-d₄) δ=9.12 (s, 1H), 9.00 (s, 2H), 7.69 (s, 1H), 7.68-7.65 (m, 1H), 7.55-7.50 (m, 2H), 7.28 (d, J=1.7 Hz, 1H), 7.24 (dd, J=2.1, 8.5 Hz, 1H), 7.07 (d, J=8.5 Hz, 1H), 6.91-6.64 (m, 1H), 3.15 (s, 3H), 2.23 (s, 3H).

### Example 4

### Synthesis of 5-amino-3-(4-(difluoromethoxy)-3-methylphenyl)-1-methyl-3-(3-(pyrimidin-5-yl)phenyl)-1,3-dihydro-2H-pyrrol-2-one (FAH-65) (1 g synthesis)

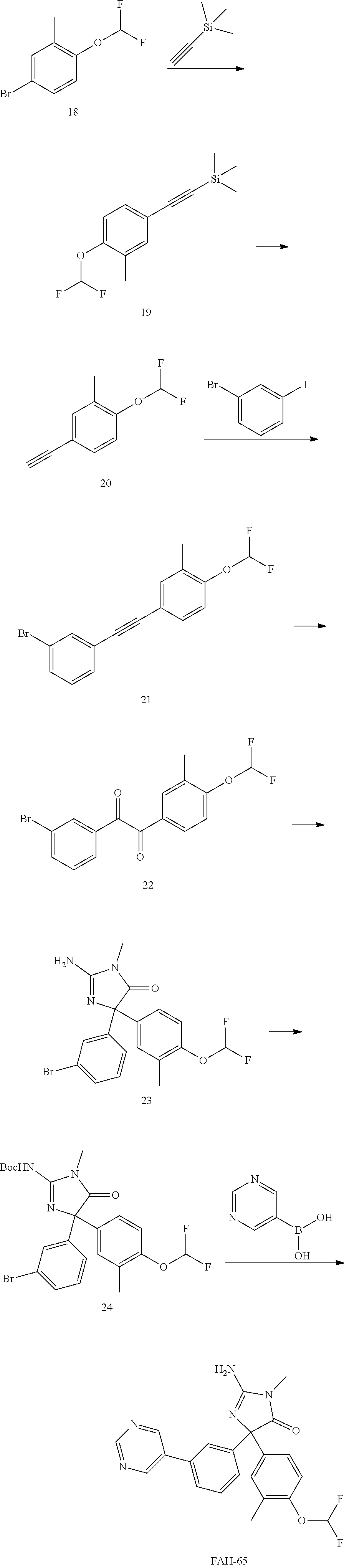

### Preparation of ((4-(difluoromethoxy)-3-methylphenyl)ethynyl)trimethylsilane (19)

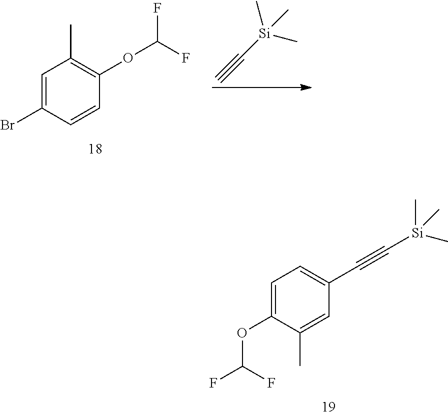

Dissolve 4-bromo-1-(difluoromethoxy)-2-methylbenzene (18) (10 g, 42.2 mmol) in dry DMF (100 mL). Trimethylsilylacetylene (7.01 mL, 50.6 mmol), and triethylamine (17.6 mL, 127 mmol) were added and the mixture was bubbled with argon gas for about 5 minutes. Copper(I)iodide (8.3 mg, 4.22 mmol) and bis(tpp)PdCl₂ (1.48 g, 2.11 mmol) were added and the mixture was stirred at 60° C. overnight. The reaction mixture was diluted with ether and washed with brine. The organic layer was separated, washed with water and dried using sodium sulfate, filtered and concentrated under reduced pressure. The crude mixture was purified using flash chromatography using hexane and ethyl acetate to yield light brown solid ((4-(difluoromethoxy)-3-methylphenyl)ethynyl)trimethylsilane (19) (5.3 g, 49.4% yield).

### Preparation of 1-(difluoromethoxy)-4-ethynyl-2-methylbenzene (20)

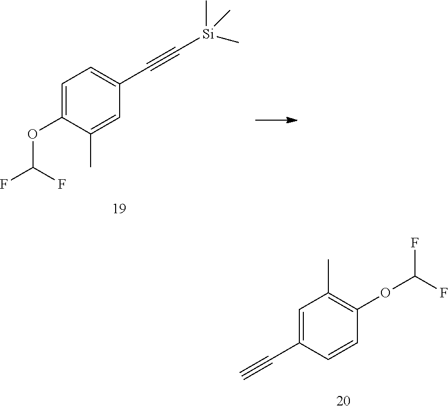 [SMILES: Cc1cc(C#C[Si](C)(C)C)ccc1OC(F)F ; C#Cc1ccc(OC(F)F)c(C)c1]

Dissolved ((4-(difluoromethoxy)-3-methylphenyl)ethynyl)trimethylsilane (19) (5.3 g, 20.8 mmol) in methanol (106 mL). Add potassium carbonate (7.2 g, 52.1 mmol) to the reaction mixture and stirred at room temperature overnight. The solids were filtered using vacuum filtration. The filtrate was diluted with 1:4 ethyl acetate:hexane and was washed with water. Water was back extracted with ethyl acetate/hexane. Organic layer was washed with brine, dried using sodium sulfate, filtered and the solvent was evaporated under reduced pressure to yield pale yellow solid 1-(difluoromethoxy)-4-ethynyl-2-methylbenzene (20) (3.5 g, 92.2% yield).

### Preparation of 4-((3-bromophenyl)ethynyl)-1-(difluoromethoxy)-2-methylbenzene (21)

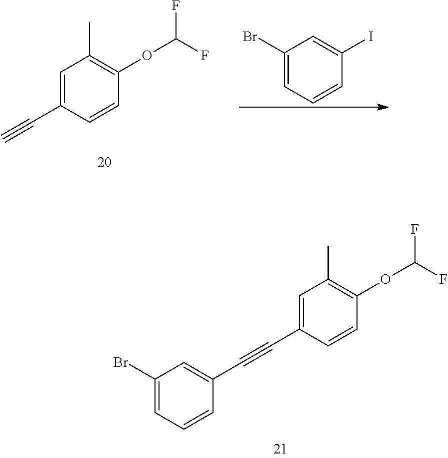 [SMILES: C#Cc1ccc(OC(F)F)c(C)c1 ; Brc1cccc(I)c1 ; Cc1cc(C#Cc2cccc(Br)c2)ccc1OC(F)F]

A solution of diisopropylethyamine (20.1 mL, 115 mmol) and DMF (28 mL), argon gas was bubbled for about 15 minutes. 1-(difluoromethoxy)-4-ethynyl-2-methylbenzene (20) (3.5 g, 19.2 mmol) and 1-bromo-3-iodobenzene (5.44 g, 19.2 mmol) were added to the reaction mixture and argon gas was bubbled for 5 minutes. Copper(I)iodide (366 mg, 1.92 mmol) and bis(tpp)PdCl₂ (674 mg, 961 μmol) were added to the reaction mixture and was stirred at 60° C. overnight. The mixture was diluted with ether and washed with water. The mixture was filtered over celite and the organic layer was separated. The aqueous layer was back extracted with ether. Combined organic layers were washed with saturated ammonium chloride solution, dried using sodium sulfate, filtered and the solvent was evaporated under reduced pressure. The crude mixture was partitioned with hexane and water. Solids were filtered, the organic layer was separated and dried using sodium sulfate. The mixture was filtered, and the solvent was evaporated under reduced pressure. The crude mixture was purified by passing through a column of silica gel using hexane as gradient to yield brown oil 4-((3-bromophenyl)ethynyl)-1-(difluoromethoxy)-2-methylbenzene (21) (6.3 g, 97.3% yield).

### Preparation of 1-(3-bromophenyl)-2-(4-(difluoromethoxy)-3-methylphenyl)ethane-1,2-dione (22)

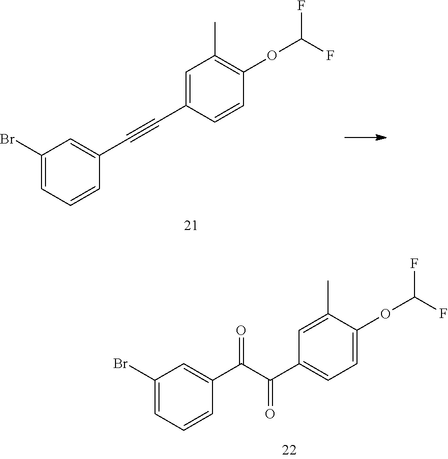 [SMILES: Cc1cc(C#Cc2cccc(Br)c2)ccc1OC(F)F ; Cc1cc(C(=O)C(=O)c2cccc(Br)c2)ccc1OC(F)F]

Dissolve 4-((3-bromophenyl)ethynyl)-1-(difluoromethoxy)-2-methylbenzene (21) (6.3 g, 18.7 mmol) in DCM (252 mL) and acetic acid (12.8 mL, 224 mmol) was added. Tetrabutylammonium bromide (9.04 g, 28 mmol) and potassium permanganate (8.86 g, 56.1 mmol) were added to the reaction mixture and was stirred at room temperature for 2 hours. The mixture was diluted with DCM and was quenched with 5% sodium metabisulphite. The organic layer was separated and dried using sodium sulfate. The crude mixture was purified using flash chromatography using hexane and ethyl acetate as gradients to yield pale yellow solid 1-(3-bromophenyl)-2-(4-(difluoromethoxy)-3-methylphenyl)ethane-1,2-dione (22) (5.4 g, 78% yield).

### Preparation of 2-amino-5-(3-bromophenyl)-5-(4-(difluoromethoxy)-3-methylphenyl)-3-methyl-3,5-dihydro-4H-imidazol-4-one (23)

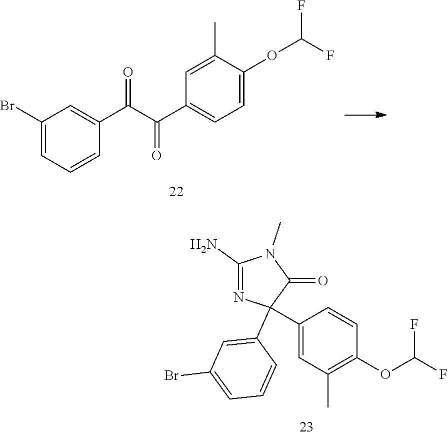 [SMILES: Cc1cc(C(=O)C(=O)c2cccc(Br)c2)ccc1OC(F)F ; Cc1cc(C2(c3cccc(Br)c3)N=C(N)N(C)C2=O)ccc1OC(F)F]

To a solution of 1-(3-bromophenyl)-2-(4-(difluoromethoxy)-3-methylphenyl)ethane-1,2-dione (22) (5.4 g, 14.6 mmol) in isopropanol (81 mL), add sodium carbonate (2.71 g, 25.6 mmol) and 1-methyl guanidine hydrochloride (2.4 g, 21.9 mmol) and was stirred at 80° C. for 2 hours. After completion of the reaction, the reaction mixture was diluted with isopropanol and chloroform and washed with water. The aqueous phase was back extracted with chloroform. The combined organic layers were dried using sodium sulfate, filtered and concentrated under reduced pressure to yield colorless solid 2-amino-5-(3-bromophenyl)-5-(4-(difluoromethoxy)-3-methylphenyl)-3-methyl-3,5-dihydro-4H-imidazol-4-one (23) (6.1 g, 98.3%).

### Preparation of tert-butyl (4-(3-bromophenyl)-4-(4-(difluoromethoxy)-3-methylphenyl)-1-methyl-5-oxo-4,5-dihydro-1H-imidazol-2-yl)carbamate (24)

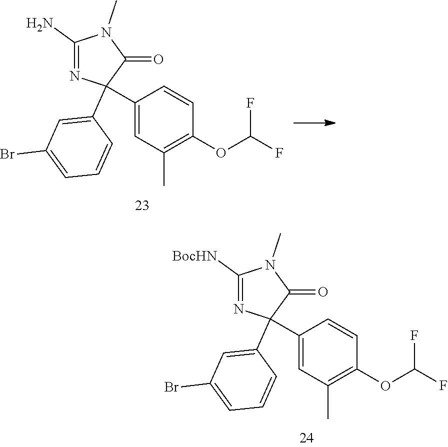 [SMILES: Cc1cc(C2(c3cccc(Br)c3)N=C(N)N(C)C2=O)ccc1OC(F)F ; Cc1cc(C2(c3cccc(Br)c3)N=C(NC(=O)OC(C)(C)C)N(C)C2=O)ccc1OC(F)F]

Dissolve 2-amino-5-(3-bromophenyl)-5-(4-(difluoromethoxy)-3-methylphenyl)-3-methyl-3,5-dihydro-4H-imidazol-4-one (23) (6.1 g, 14.4 mmol) in DCM (61 mL) and cool it to 0 C. Di-tert-butyl-dicarbonate (3.29 g, 15.1 mmol) was added to the reaction mixture and was stirred at room temperature overnight. After completion of the reaction, the reaction mixture was concentrated under reduced pressure. The crude mixture was purified using flash chromatography using hexane and ethyl acetate as gradients to yield off-white solid tert-butyl (4-(3-bromophenyl)-4-(4-(difluoromethoxy)-3-methylphenyl)-1-methyl-5-oxo-4,5-dihydro-1H-imidazol-2-yl)carbamate (24) (3.5 g, 46.4%).

### Synthesis of 2-amino-5-(4-(difluoromethoxy)-3-methylphenyl)-3-methyl-5-(3-(pyrimidin-5-yl)phenyl)-3,5-dihydro-4H-imidazol-4-one (FAH-65)

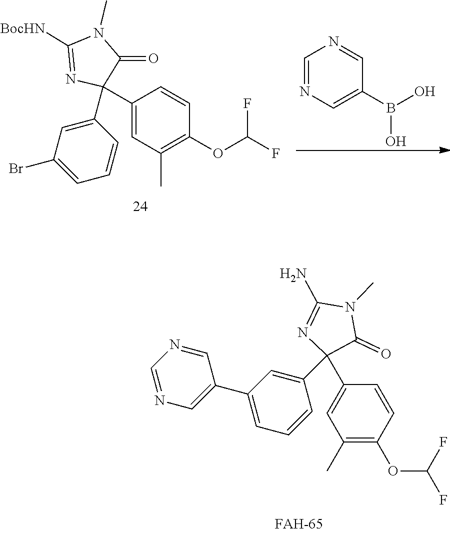 [SMILES: Cc1cc(C2(c3cccc(Br)c3)N=C(NC(=O)OC(C)(C)C)N(C)C2=O)ccc1OC(F)F ; OB(O)c1cncnc1 ; Cc1cc(C2(c3cccc(-c4cncnc4)c3)N=C(N)N(C)C2=O)ccc1OC(F)F]

Dissolved tert-butyl (4-(3-bromophenyl)-4-(4-(difluoromethoxy)-3-methylphenyl)-1-methyl-5-oxo-4,5-dihydro-1H-imidazol-2-yl)carbamate (24) (2.0 g, 3.81 mmol) in dioxane (10 mL). Pyrimidine-5-boronic acid (473 mg, 3.81 mmol), 2 M potassium carbonate solution (5.82 mL) and Xphos Palladacycle Gen.2 (300 mg, 381 μmol) were added and the mixture was heated at 110° C. for 2 h in a N₂ flushed sealed vial on a hot plate. The reaction mixture was partitioned with water and chloroform and further extracted to obtain the product. The combined organic layers were washed with brine, dried using sodium sulfate, and concentrated under reduced pressure. On an 80 g silica column, the crude was loaded and eluted with 0-30% ACN/DCM (10 CV) then 2 CV at 30% to get the Boc-protected compound. After concentrating down, the fraction was dissolved in DCM and added 1 g of charcoal, stirred for 10 min, filtered through a Celite plug and concentrated to obtain a pale-yellow solid (1.4 g). Then continued to elute the column with 10% MeOH/DCM to get de-Boc product. After concentrating down, the fraction was dissolved in MeOH/DCM and added charcoal, stirred for 10 min, filtered through a Celite plug and concentrated to obtain 350 mg of the title compound.

The Boc-product (1.4 g) was dissolved in DCM and TFA was added. The mixture was stirred at 40° C. for 2 h. The reaction was concentrated, and partitioned in between EA and Na₂CO₃ solution. The organic layer was washed with brine, dried over MgSO4, concentrated, and triturated with DCM to give an off-white solid (820 mg). The multiple crops of product were combined in acetonitrile and DCM in 20 ml vial and concentrated to obtain a uniform solid for characterization. ¹H NMR (600 MHz, Methanol-d₄) δ =9.12 (s, 1H), 9.00 (s, 2H), 7.69 (s, 1H), 7.68-7.65 (m, 1H), 7.55-7.50 (m, 2H), 7.28 (d, J=1.7 Hz, 1H), 7.24 (dd, J=2.1, 8.5 Hz, 1H), 7.07 (d, J=8.5 Hz, 1H), 6.91-6.64 (m, 1H), 3.15 (s, 3H), 2.23 (s, 3H). LCMS (+esi) [M+H]⁺=424.4.

### Example 5

### Protocol for Enantiomer Separation

### Protocol for Enantiomer Separation of FAH-65 Using Supercritical Fluid Chromatography

Preparative Method: IC (2×25 cm), 40% isopropanol (0.1% DEA)/CO₂, 100 bar, 50 mL/min, 220 nm, inj vol.: 1 mL, 20 mg/mL ethanol:DCM. Analytical Method: IC (25×0.46 cm), 40% isopropanol (DEA)/CO₂, 120 bar, 3 mL/min, 220, 254 and 280 nm. Separation of 1 g of FAH-65 yielded 525 mg of peak-1 and 465 mg of peak-2 (see FIGS. 20, 21, and 22). FAH-65 Peak-1: [α]27.0D -180.0° (c=1.00, CH₂Cl₂); FAH-65 Peak-2: [α]26.6D+120.0° (c=1.00, CH₂Cl₂)

### Example 6

### Biological Assay

### P5-P5′ Data

For the assay, 4 μL of assay buffer was added to each well, followed by 2 μL of BACE-1 diluted in assay buffer to 7.5 ng/μL. Then, 2 μL of inhibitor, at concentrations along an 11 pt. two-fold dilution series starting at 0.5 μM, were added to appropriate wells and incubated for 30 minutes at room temperature. Afterwards, 2 μL of P5-P5′ BACE-1 substrate, diluted to 50 μM in assay buffer, were added to each well, after which the signal generated was read every 30 minutes at 25° C. for 2 hr. Signals shown in FIG. 15 are representative of readings at 2 hrs.

### Cathepsin-D Data

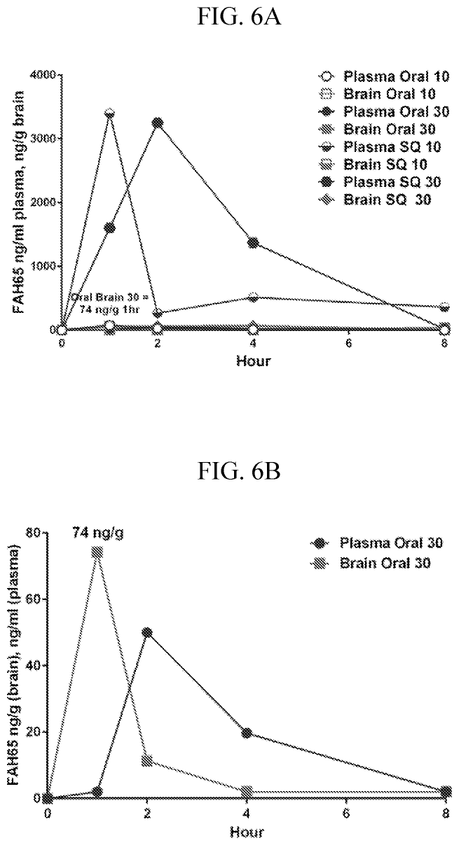

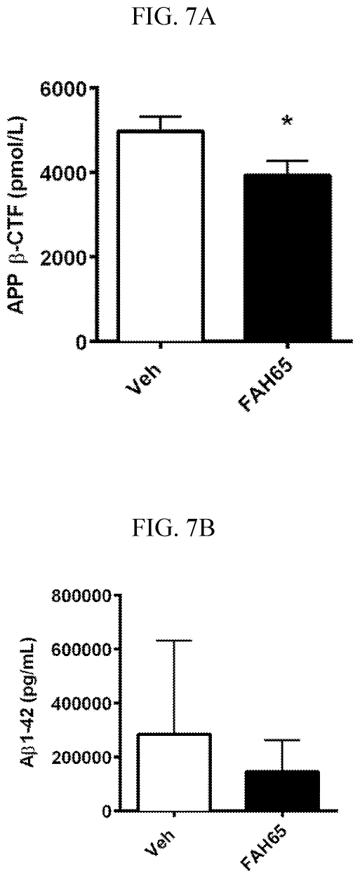

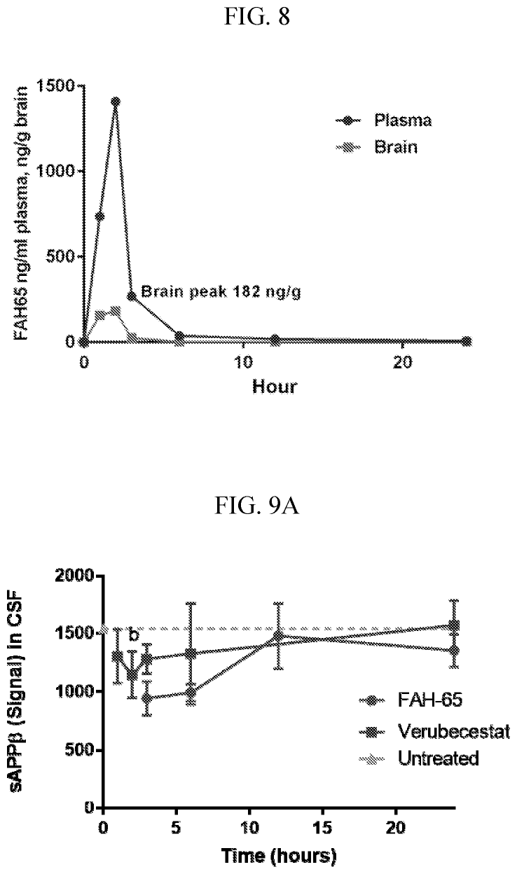

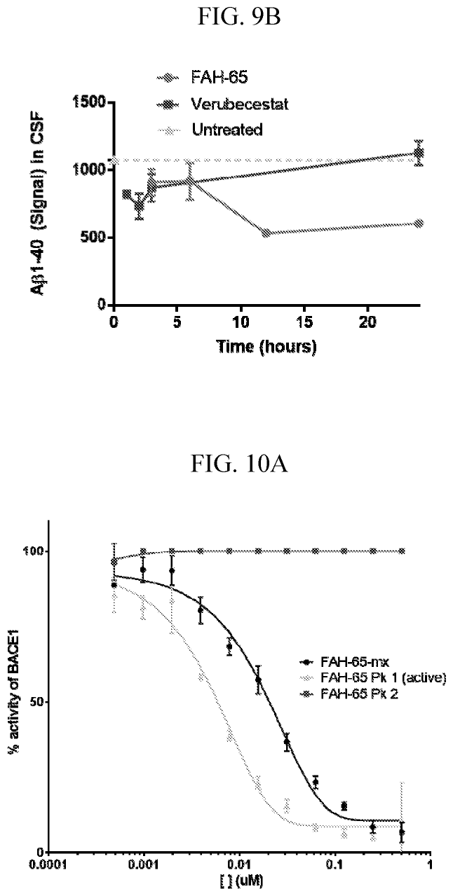

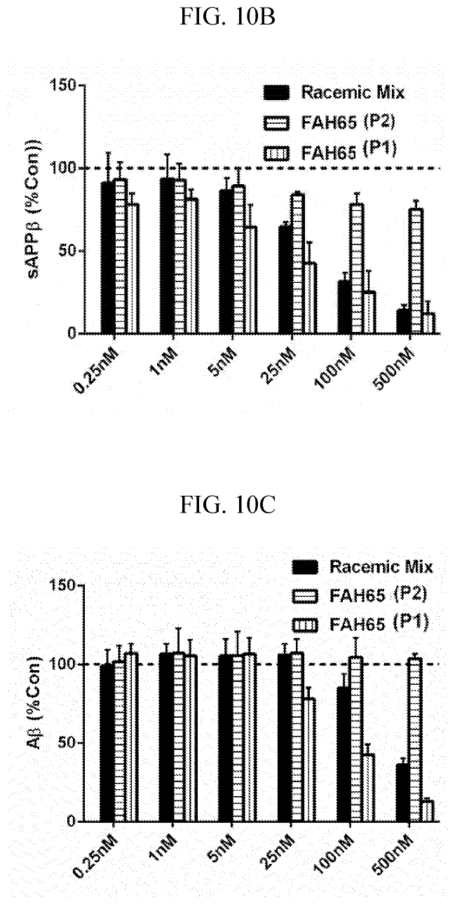

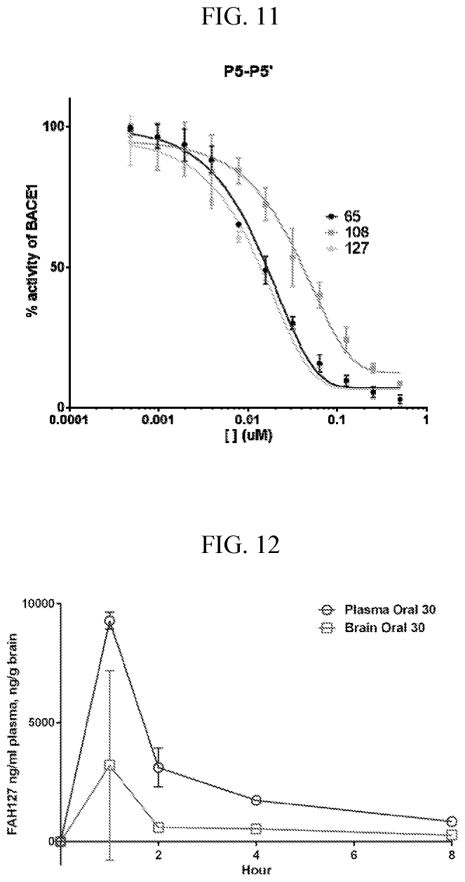

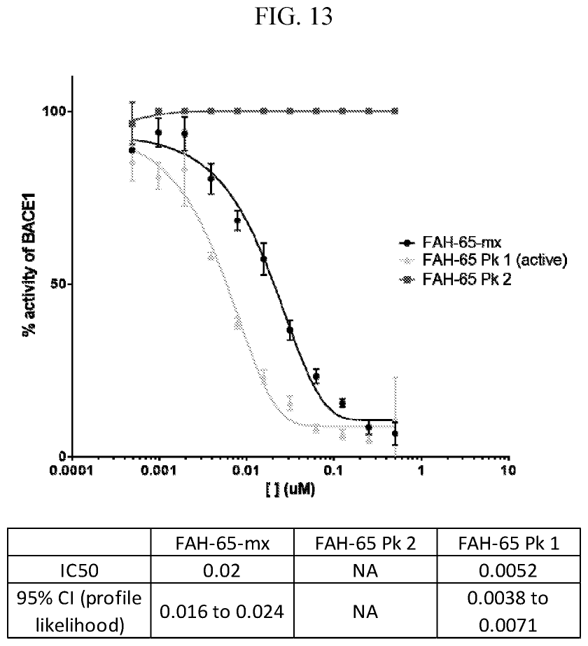

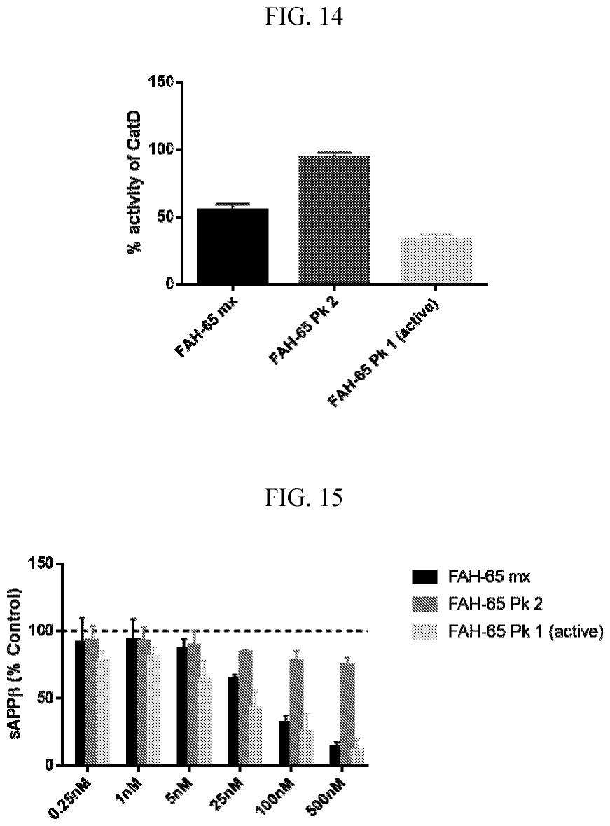

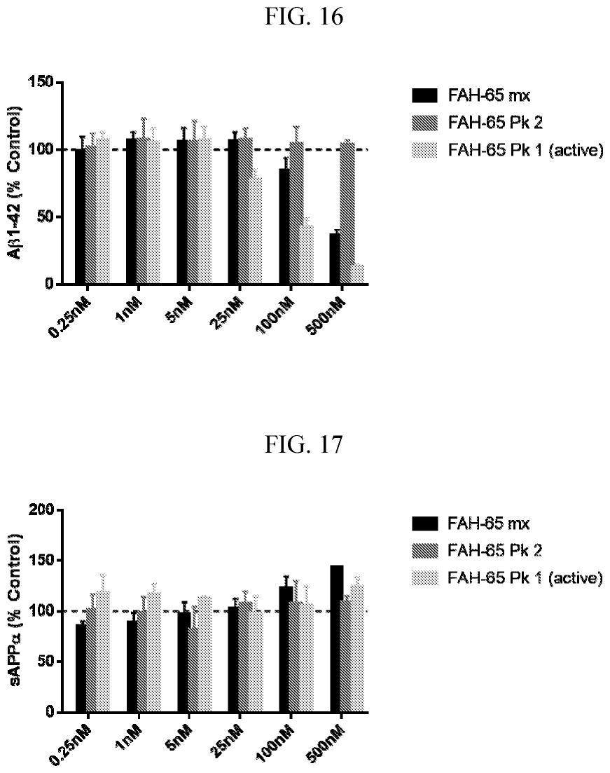

For the assay, 4 μL of assay buffer was added to each well, followed by 2 μL of Cathepsin D (CatD) diluted in assay buffer to 5 ng/μL. Then, 2 μL of inhibitor, prepared to get a testing concentration of 20 μM, were added to appropriated wells and incubated for 30 minutes at room temperature. Afterwards, 2 μL of CatD substrate, diluted to 50 μM in assay buffer, were added to each well, after which the signal generated was read every 30 minutes at 25° C. for 1 hr. Signals shown in FIG. 6 are representative of readings at 1 hr.

### CHO-7W Cell Data

For the assay, 50 μL of CHO-7W cells were added to each well at 4×10\hat{ }5 cells/mL. Then, 50 μL of inhibitor, prepared for tested concentrations, were added to appropriated wells and incubated for 24 hours at 37° C. Afterwards, media from each well is collected and assayed using an AlphaLISA assay for respective analytes.

### NRG1 and PSGLS1 Assay

293T cells were plated in 96-well plates at 40,000 cells/well, incubated overnight at 37° C. and 5% CO₂. Next day, cells were co-transfected with NRG1-BACE1 plasmids or PSGL1-BACE1 plasmids using Lipofectamine2000, as describe previously. Six hours after transfection, the reagents were removed and media containing the different compounds at various concentrations were loaded into each well.

After overnight incubation with the compounds, 20 μL of media were removed and added to a new plate. Then, 200 μl of reaction solution (0.1 M glycine, pH 10.4, 1 mM MgCl2, 1 mM ZnCl2 containing 1 mg/ml 4-nitrophenyl phosphate disodium salt hexahydrate, Sigma S0942) were loaded into each well of the plate. The absorbance was read at 405 nm for 60 min in 30 min intervals in a SpectraMax M5.

BACE inhibitor IV was purchased from EMD Millipore (cat 565788) and Verubecestat was purchased from Selleckchem (cat S8564). These compounds were diluted to 10 mM in DMSO. This solution was diluted to 20, 10, 2, 1, 0.2 and 0.02 μM in DMEM high Glucose containing 10% of FBS and 1% of Penicillin-Streptomycin containing 0.2% DMSO.

TABLE 1
IC₅₀ values in a cell-free BACE assay for representative ASBIs.

| FAH# | Molecular Weight | BACE-1 IC50 (nM) |
|---|---|---|
| 65 | 469.45 | 22 |
| 74 | 409.4 | 13 |
| 84 | 391.14 | 51 |
| 86 | 469.45 | 18 |
| 108 | 394.39 | 39 |
| 110 | 484.17 | 43 |
| 127 | 441.41 | 13 |

TABLE 2
ASBI Compounds and Experimental Results

| | | | | | BACE1 | CatD IC50 | |
|---|---|---|---|---|---|---|---|
| Name | Structure | MW | cLogP | TPSA | IC50 (μM) | (μM) | PAMPA |
| FAH-31 | 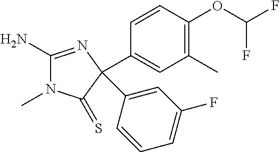 [SMILES: Cc1cc(C2(c3cccc(F)c3)N=C(N)N(C)C2=S)ccc1OC(F)F] | 379.40 | 2.76 | 50.85 | 4.34 3.96 | | 3.07 |
| FAH-32 | 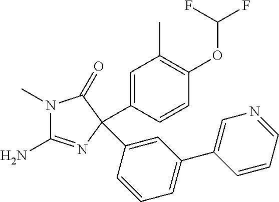 [SMILES: Cc1cc(C2(c3cccc(-c4cccnc4)c3)N=C(N)N(C)C2=O)ccc1OC(F)F] | 422.40 | 2.67 | 80.81 | 0.07 0.05 0.011 0.016 0.017 | >100 | 0.91 0.92 |
| FAH-33 | 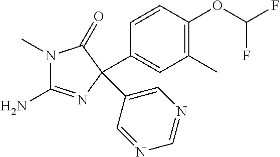 [SMILES: Cc1cc(C2(c3cncnc3)N=C(N)N(C)C2=O)ccc1OC(F)F] | 347.30 393.34 | 1.70 | 93.70 | 10 >5 | | 0.24 |
| FAH-37 | 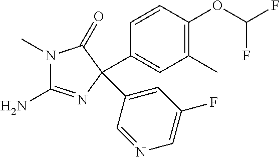 [SMILES: Cc1cc(C2(c3cncc(F)c3)N=C(N)N(C)C2=O)ccc1OC(F)F] | 364.30 | 1.67 | 80.81 | 1.93 2.8 5.3 4.7 | | 0.54 |
| FAH-38 | 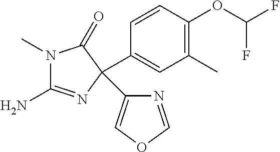 [SMILES: Cc1cc(C2(c3cocn3)N=C(N)N(C)C2=O)ccc1OC(F)F] | 336.30 382.32 | 1.47 | 93.95 | 15.26 >5 | | 3.46 |
| FAH-42 | 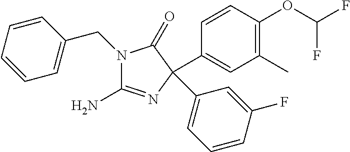 [SMILES: Cc1cc(C2(c3cccc(F)c3)N=C(N)N(Cc3ccccc3)C2=O)ccc1OC(F)F] | 439.40 | 3.91 | 67.92 | 2.3 2.7 | | 2.29 |
| FAH-43 | 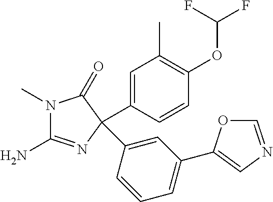 [SMILES: Cc1cc(C2(c3cccc(-c4cnco4)c3)N=C(N)N(C)C2=O)ccc1OC(F)F] | 412.40 | 2.55 | 93.95 | 0.14 0.23 0.49 | | 0.79 |
| FAH-44 | 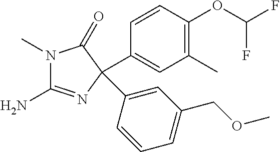 [SMILES: COCc1cccc(C2(c3ccc(OC(F)F)c(C)c3)N=C(N)N(C)C2=O)c1] | 389.40 | 2.17 | 77.15 | 0.3 0.05 | | 0.91 |
| FAH-46 | 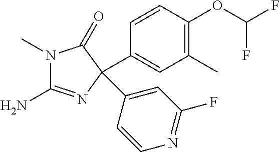 [SMILES: Cc1cc(C2(c3ccnc(F)c3)N=C(N)N(C)C2=O)ccc1OC(F)F] | 364.30 | 1.67 | 80.81 | 1.6 2.99 | | 0.65 |
| FAH-47 | 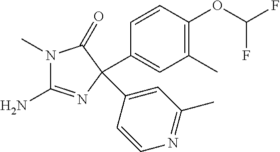 [SMILES: Cc1cc(C2(c3ccc(OC(F)F)c(C)c3)N=C(N)N(C)C2=O)ccn1] | 360.40 | 1.91 | 80.81 | 1.4 3.05 | | 0.46 |
| FAH-48 | 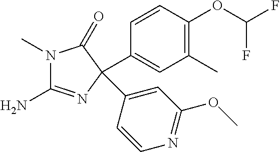 [SMILES: COc1cc(C2(c3ccc(OC(F)F)c(C)c3)N=C(N)N(C)C2=O)ccn1] | 376.40 | 1.51 | 90.04 | 4.8 4.35 | | 0.68 |
| FAH-53 | 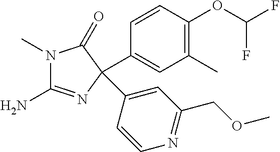 [SMILES: COCc1cc(C2(c3ccc(OC(F)F)c(C)c3)N=C(N)N(C)C2=O)ccn1] | 390.40 | 1.57 | 90.04 | 1.900 | | 0.26 |
| FAH-57 | 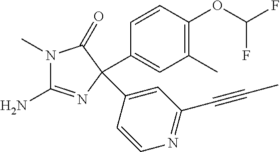 [SMILES: CC#Cc1cc(C2(c3ccc(OC(F)F)c(C)c3)N=C(N)N(C)C2=O)ccn1] | 384.40 | 1.73 | 80.81 | 0.24 0.44 0.51 | | 0.61 |
| FAH-59 | 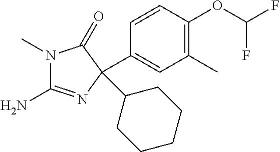 [SMILES: Cc1cc(C2(C3CCCCC3)N=C(N)N(C)C2=O)ccc1OC(F)F] | 351.40 397.42 | 2.87 | 67.92 | 1.5 0.75 0.72 | | 4.43 |
| FAH-62 | 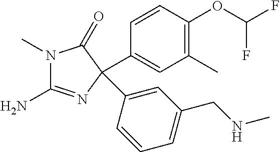 [SMILES: CNCc1cccc(C2(c3ccc(OC(F)F)c(C)c3)N=C(N)N(C)C2=O)c1] | 388.40 480.46 | 1.86 | 79.95 | 0.370 | | 0.35 |
| FAH-63 | 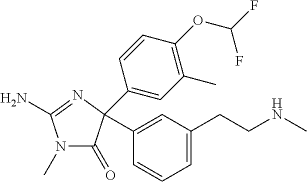 [SMILES: CNCCc1cccc(C2(c3ccc(OC(F)F)c(C)c3)N=C(N)N(C)C2=O)c1] | 402.40 494.49 | 2.02 | 79.95 | 1.180 | | 0.38 |
| FAH-64 | 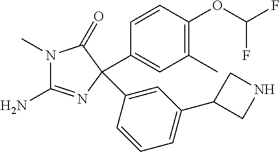 [SMILES: Cc1cc(C2(c3cccc(C4CNC4)c3)N=C(N)N(C)C2=O)ccc1OC(F)F] | 400.40 492.48 | 1.90 | 79.95 | 3.200 | | 0.35 |
| FAH-65 | 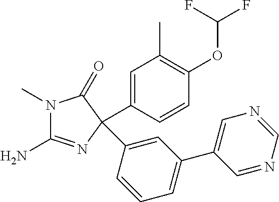 [SMILES: Cc1cc(C2(c3cccc(-c4cncnc4)c3)N=C(N)N(C)C2=O)ccc1OC(F)F] | 423.4 469.45 | 2.79 | 93.70 | 0.019 0.016 0.011 0.015 0.024 0.014 NA Pk1 0.0052 Pk2 0.0079 Pk2 0.01116 | 47.06 | 0.44 0.45 0.46 0.47 0.52 0.48 0.49 |
| FAH-66 | 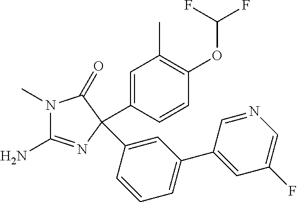 [SMILES: Cc1cc(C2(c3cccc(-c4cncc(F)c4)c3)N=C(N)N(C)C2=O)ccc1OC(F)F] | 440.40 | 2.74 | 80.81 | 0.018 0.12 0.012 0.019 0.047 0.028 0.036 | | 1.15 |
| FAH-67 | 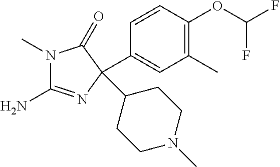 [SMILES: Cc1cc(C2(C3CCN(C)CC3)N=C(N)N(C)C2=O)ccc1OC(F)F] | 366.40 | 1.53 | 71.16 | 24.250 | | 0.20 |
| FAH-68 | 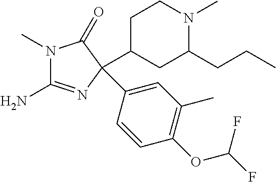 [SMILES: CCCC1CC(C2(c3ccc(OC(F)F)c(C)c3)N=C(N)N(C)C2=O)CCN1C] | 408.50 | 2.51 | 71.16 | 16.100 | | 0.20 |
| FAH-69 | 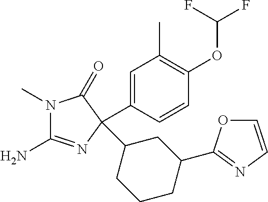 [SMILES: Cc1cc(C2(C3CCCC(c4ncco4)C3)N=C(N)N(C)C2=O)ccc1OC(F)F] | 418.40 | 2.99 | 93.95 | 0.11 0.21 0.12 0.43 | | 0.92 |
| FAH-72 | 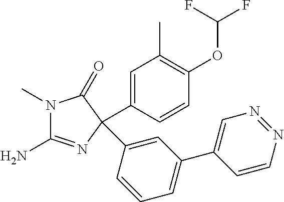 [SMILES: Cc1cc(C2(c3cccc(-c4ccnnc4)c3)N=C(N)N(C)C2=O)ccc1OC(F)F] | 423.40 469.45 | 2.79 | 93.70 | 0.14 0.10 0.049 s 0.087 s 0.093 s | | 0.37 |
| FAH-73 | 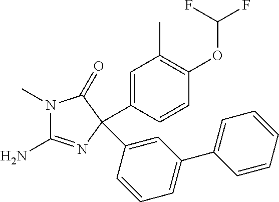 [SMILES: Cc1cc(C2(c3cccc(-c4ccccc4)c3)N=C(N)N(C)C2=O)ccc1OC(F)F] | 421.45 467.47 | 3.31 | 67.92 | 0.87 0.53 s 0.69 s | | 5.11 |
| FAH-74 | 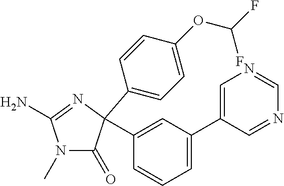 [SMILES: CN1C(=O)C(c2ccc(OC(F)F)cc2)(c2cccc(-c3cncnc3)c2)N=C1N] | 409.40 455.42 | 2.48 | 93.70 | 0.009 0.0042 0.0025 0.0057 0.0075 0.015 0.009 0.023 | | 0.36 |
| FAH-75 | 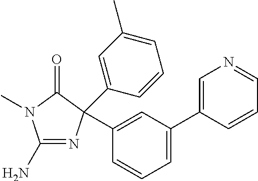 [SMILES: Cc1cccc(C2(c3cccc(-c4cccnc4)c3)N=C(N)N(C)C2=O)c1] | 357.40 | 2.58 | 84.47 | 0.21 0.24 0.15 0.42 | | 0.62 |
| FAH-82 | 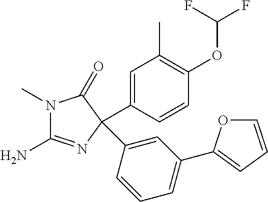 [SMILES: Cc1cc(C2(c3cccc(-c4ccco4)c3)N=C(N)N(C)C2=O)ccc1OC(F)F] | 411.40 457.43 | 2.89 | 81.06 | 0.78 0.45 2.40 | | 3.06 3.06 |
| FAH-83 | 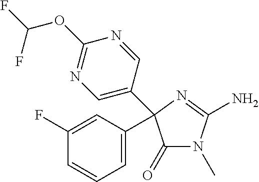 [SMILES: CN1C(=O)C(c2cnc(OC(F)F)nc2)(c2cccc(F)c2)N=C1N] | 351.30 397.31 | 1.57 | 93.70 | 2.91 2.33 9.19 | | 0.51 0.53 |
| FAH-84 | 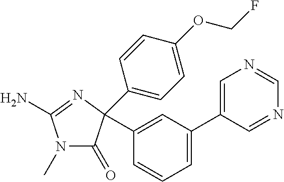 [SMILES: CN1C(=O)C(c2ccc(OCF)cc2)(c2cccc(-c3cncnc3)c2)N=C1N] | 391.14 437.15 | 2.21 | 93.70 | 0.028 0.097 | | 0.30 |
| FAH-85 | 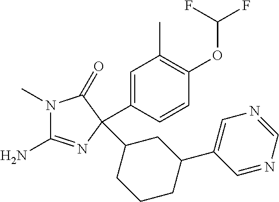 [SMILES: Cc1cc(C2(C3CCCC(c4cncnc4)C3)N=C(N)N(C)C2=O)ccc1OC(F)F] | 429.50 | 3.28 | 93.70 | 0.220 | | 0.51 2 peaks |
| FAH-86 | 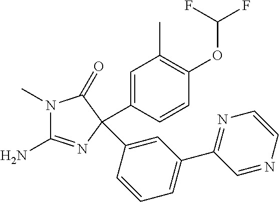 [SMILES: Cc1cc(C2(c3cccc(-c4cnccn4)c3)N=C(N)N(C)C2=O)ccc1OC(F)F] | 423.40 469.45 | 2.79 | 93.70 | 0.014 s 0.016 s 0.009 0.028 | >50 | 0.68 |
| FAH-87 | 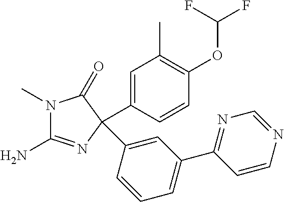 [SMILES: Cc1cc(C2(c3cccc(-c4ccncn4)c3)N=C(N)N(C)C2=O)ccc1OC(F)F] | 423.40 469.45 | 2.79 | 93.70 | 0.30 s 0.34 s 0.42 s 0.29 | | 0.58 |
| FAH-88 | 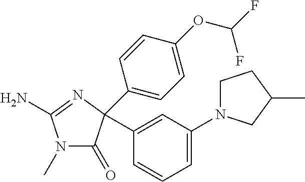 [SMILES: CC1CCN(c2cccc(C3(c4ccc(OC(F)F)cc4)N=C(N)N(C)C3=O)c2)C1] | 414.45 | 2.56 | 71.16 | 0.84 0.60 2.13 | | 4.53 2-peaks |
| FAH-99 | 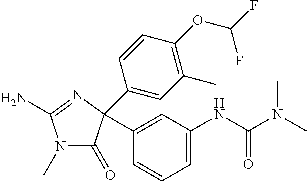 [SMILES: Cc1cc(C2(c3cccc(NC(=O)N(C)C)c3)N=C(N)N(C)C2=O)ccc1OC(F)F] | 431.4 477.47 | 1.80 | 100.30 | 0.15 0.21 | | 0.38 |
| FAH-100 | 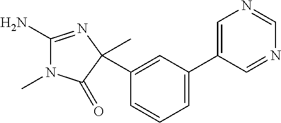 [SMILES: CN1C(=O)C(C)(c2cccc(-c3cncnc3)c2)N=C1N] | 281.3 327.32 | 1.68 | 84.47 | 2.31 s 4.85 | | 0.27 |
| FAH-101 | 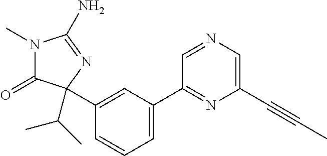 [SMILES: CC#Cc1cncc(-c2cccc(C3(C(C)C)N=C(N)N(C)C3=O)c2)n1] | 347.4 393.45 | 2.56 | 84.47 | assay problem | | 1.71 |
| FAH-106 | 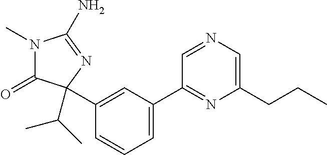 [SMILES: CCCc1cncc(-c2cccc(C3(C(C)C)N=C(N)N(C)C3=O)c2)n1] | 351.4 384.46 | 3.26 | 84.47 | 2.18 3.164 | | 2.36 |
| FAH-107 | 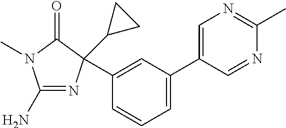 | 321.38 367.41 | 2.29 | 84.47 | 8.87 4.93 6.88 | | 0.40 |
| FAH-110 | 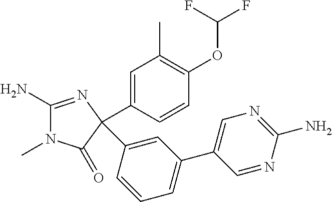 [SMILES: Cc1cc(C2(c3cccc(-c4cnc(N)nc4)c3)N=C(N)N(C)C2=O)ccc1OC(F)F] | 438.44 484.17 | 2.34 | 119.70 | 0.047 0.038 | | 0.46 |
| FAH-111 | 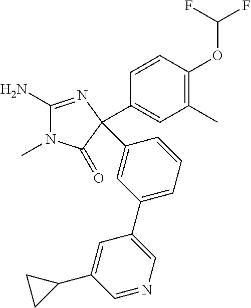 [SMILES: Cc1cc(C2(c3cccc(-c4cncc(C5CC5)c4)c3)N=C(N)N(C)C2=O)ccc1OC(F)F] | 462.5 508.53 | 3.30 | 80.81 | 0.08 0.11 | | solubility issue |
| FAH-116 | 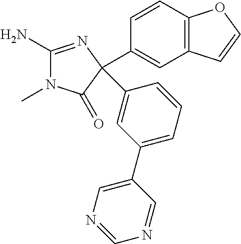 [SMILES: CN1C(=O)C(c2cccc(-c3cncnc3)c2)(c2ccc3occc3c2)N=C1N] | 383.40 | 2.71 | 97.61 | 0.27 0.26 | | 0.52 |
| FAH-117 | 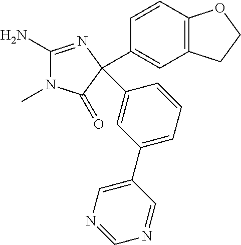 [SMILES: CN1C(=O)C(c2cccc(-c3cncnc3)c2)(c2ccc3c(c2)CCO3)N=C1N] | 385.40 | 2.31 | 93.70 | 1.37 0.41 | | 0.32 |
| FAH-126 | 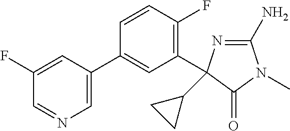 [SMILES: CN1C(=O)C(c2cc(-c3cncc(F)c3)ccc2F)(C2CC2)N=C1N] | 342.30 | 1.93 | 71.58 | inactive | | |
| FAH-127 | 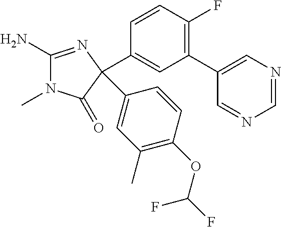 [SMILES: Cc1cc(C2(c3ccc(F)c(-c4cncnc4)c3)N=C(N)N(C)C2=O)ccc1OC(F)F] | 441.40 | 2.85 | 93.70 | 0.014 0.013 0.005 0.0246 | | 0.53 0.49 0.53 0.50 |
| FAH-135 | 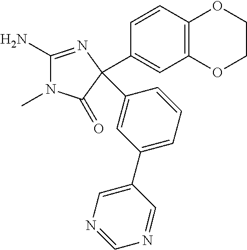 [SMILES: CN1C(=O)C(c2cccc(-c3cncnc3)c2)(c2ccc3c(c2)OCCO3)N=C1N] | 401.4 | 1.95 | 102.90 | 0.21 | | 0.26 |
| FAH-136 | 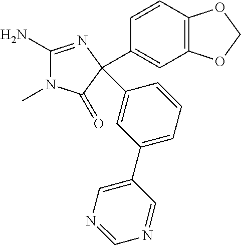 [SMILES: CN1C(=O)C(c2cccc(-c3cncnc3)c2)(c2ccc3c(c2)OCO3)N=C1N] | 387.4 | 1.90 | 102.90 | 0.25 | | 0.30 |
| FAH-137 | 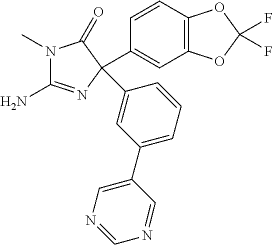 [SMILES: CN1C(=O)C(c2cccc(-c3cncnc3)c2)(c2ccc3c(c2)OC(F)(F)O3)N=C1N] | 423.4 | 2.14 | 102.90 | 0.46 | | 0.59 |
| FAH-152 | 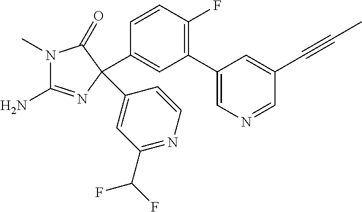 [SMILES: CC#Cc1cncc(-c2cc(C3(c4ccnc(C(F)F)c4)N=C(N)N(C)C3=O)ccc2F)c1] | 449.40 | 2.20 | 84.47 | 0.02 | | 0.46 |
| FAH-156 | 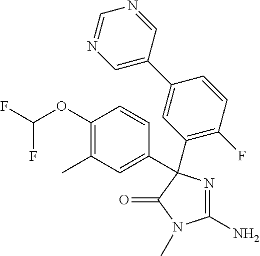 [SMILES: Cc1cc(C2(c3cc(-c4cncnc4)ccc3F)N=C(N)N(C)C2=O)ccc1OC(F)F] | 441.40 | 2.85 | 93.70 | 0.20 | | 0.25 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of formula (I): 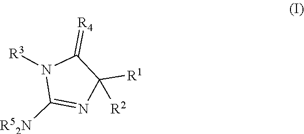
or a pharmaceutically acceptable salt thereof, wherein:
R¹ is alkyl, heteroalkyl, cycloalkyl, heterocyclyl, heterocyclylalkyl, aryl, aralkyl, heteroaryl, heteroarylalkyl, aryl-aryl, aryl-heteroaryl, NR³(CO)R⁶, OR⁶ or CO₂R⁶;
R² is alkyl, heteroalkyl, cycloalkyl, heterocyclyl, heterocyclylalkyl, aryl, aralkyl, heteroaryl, heteroarylalkyl, OR⁶, CO₂R⁶, NR³(CO)R⁶, or (CO)NR³R⁶ wherein R³ and R⁶, taken together with the N to which they are attached, may form a heterocyclyl or heteroaryl moiety;
each R³ is independently hydrogen, alkyl, aryl or aralkyl;
R⁴ is O, S or NR³;
each R⁵ is independently H or alkyl;
each R⁶ is independently H, alkyl, heteroalkyl, cycloalkyl, heterocyclyl, heterocyclylalkyl, aryl, aralkyl, heteroaryl, heteroarylalkyl, alkoxy or fluoroalkoxy.

2. The compound of claim 1, wherein R¹ is aryl-aryl or aryl-heteroaryl.

3. The compound of claim 1 or 2, wherein R¹ is aryl-heteroaryl.

4. The compound of claim 1, wherein R¹ is aryl, heteroaryl or cycloalkyl.

5. The compound of claim 1, wherein R¹ is: 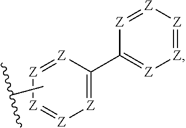
wherein:
each Z is independently C—R⁷ or N; and
each R⁷ is independently H, alkyl, halo, heteroalkyl, cycloalkyl, heterocyclyl, heterocyclylalkyl, aryl, aralkyl, heteroaryl, heteroarylalkyl, alkoxy, fluoroalkoxy, OR⁶, NR⁶, CN, CO₂R⁶, NR³(CO)R⁶, or (CO)NR³R⁶ wherein R³ and R⁶, taken together with the N to which they are attached, may form a heterocyclyl, or heteroaryl moiety.

6. The compound of claim 5, wherein four Z are N.

7. The compound of claim 5, wherein three Z are N.

8. The compound of claim 5, wherein two Z are N.

9. The compound of any one of claims 1-3, 5, 8, wherein R¹ is:

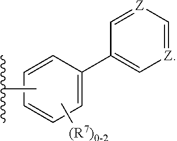

10. The compound of any one of claims 1-3, 5, 8, 9, wherein R¹ is:

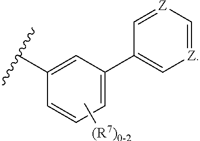

11. The compound of any one of claims 1-3, 5, 8-10, wherein R¹ is:

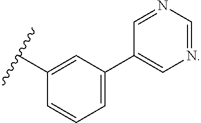 [SMILES: *c1cccc(-c2cncnc2)c1]

12. The compound of any one of claims 1-3, 5, 9-10, wherein R¹ is:

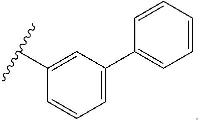 [SMILES: *c1cccc(-c2ccccc2)c1]

13. The compound of any one of claims 1-3, 5, 9-10, wherein R¹ is: 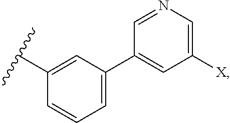
wherein X is F, Cl, Br, or I.

14. The compound of claim 13, wherein X is F.

15. The compound of any one of claims 1-3, 5, 8-10, wherein R¹ is:

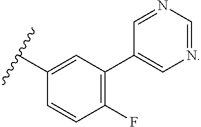 [SMILES: *c1ccc(F)c(-c2cncnc2)c1]

16. The compound of any one of claims 1-15, wherein R¹is aryl or heteroaryl.

17. The compound of any one of claims 1-16, wherein R² is aryl.

18. The compound of any one of claims 1-17, wherein R² is substituted phenyl.

19. The compound of any one of claims 1-18, wherein R² is:

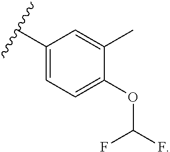 [SMILES: *c1ccc(OC(F)F)c(C)c1]

20. The compound of any one of claims 1-19, wherein R³ is alkyl.

21. The compound of any one of claims 1-20, wherein R³ is methyl.

22. The compound of any one of claims 1-21, wherein R⁴ is O.

23. The compound of any one of claims 1-22, wherein each R⁵ is H.

24. A compound selected from the list consisting of: 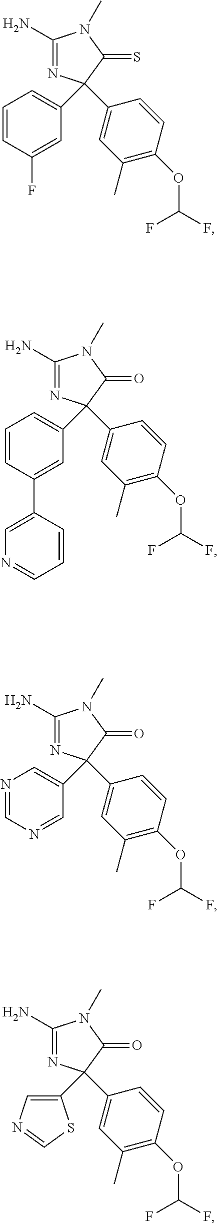 [SMILES: Cc1cc(C2(c3cccc(F)c3)N=C(N)N(C)C2=S)ccc1OC(F)F ; Cc1cc(C2(c3cccc(-c4cccnc4)c3)N=C(N)N(C)C2=O)ccc1OC(F)F ; Cc1cc(C2(c3cncnc3)N=C(N)N(C)C2=O)ccc1OC(F)F ; Cc1cc(C2(c3cncs3)N=C(N)N(C)C2=O)ccc1OC(F)F] 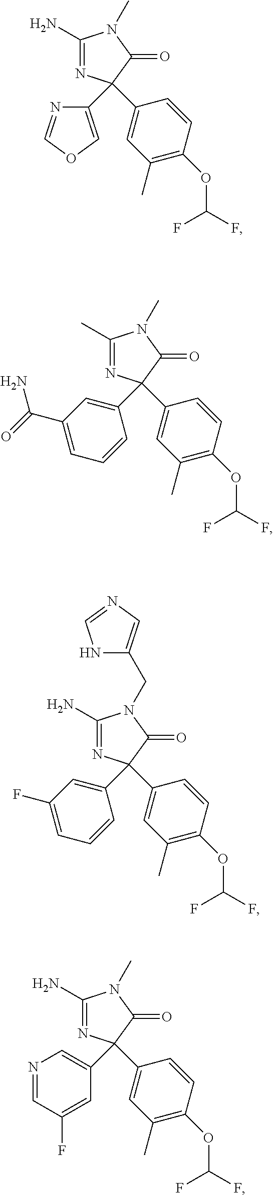 [SMILES: Cc1cc(C2(c3cocn3)N=C(N)N(C)C2=O)ccc1OC(F)F ; CC1=NC(c2cccc(C(N)=O)c2)(c2ccc(OC(F)F)c(C)c2)C(=O)N1C ; Cc1cc(C2(c3cccc(F)c3)N=C(N)N(Cc3cnc[nH]3)C2=O)ccc1OC(F)F ; Cc1cc(C2(c3cncc(F)c3)N=C(N)N(C)C2=O)ccc1OC(F)F] 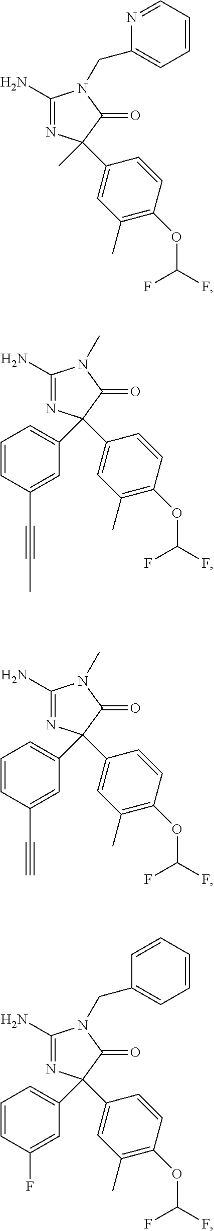 [SMILES: Cc1cc(C2(C)N=C(N)N(Cc3ccccn3)C2=O)ccc1OC(F)F ; CC#Cc1cccc(C2(c3ccc(OC(F)F)c(C)c3)N=C(N)N(C)C2=O)c1 ; C#Cc1cccc(C2(c3ccc(OC(F)F)c(C)c3)N=C(N)N(C)C2=O)c1 ; Cc1cc(C2(c3cccc(F)c3)N=C(N)N(Cc3ccccc3)C2=O)ccc1OC(F)F] 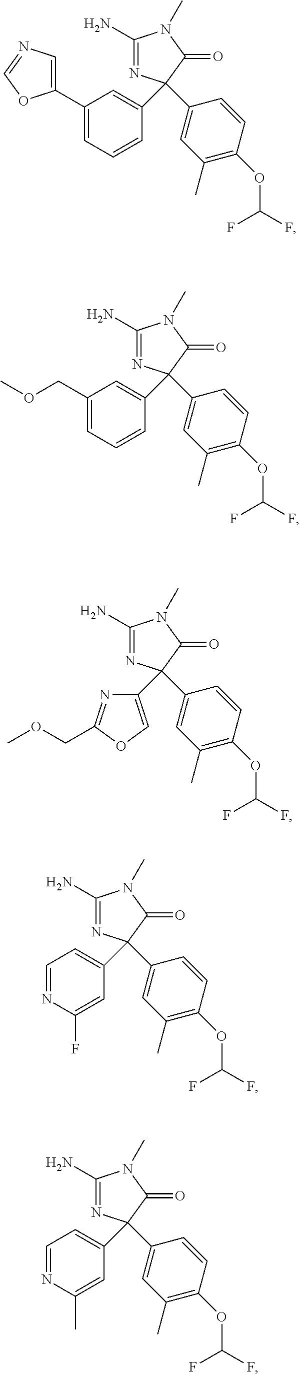 [SMILES: Cc1cc(C2(c3cccc(-c4cnco4)c3)N=C(N)N(C)C2=O)ccc1OC(F)F ; COCc1cccc(C2(c3ccc(OC(F)F)c(C)c3)N=C(N)N(C)C2=O)c1 ; COCc1nc(C2(c3ccc(OC(F)F)c(C)c3)N=C(N)N(C)C2=O)co1 ; Cc1cc(C2(c3ccnc(F)c3)N=C(N)N(C)C2=O)ccc1OC(F)F ; Cc1cc(C2(c3ccc(OC(F)F)c(C)c3)N=C(N)N(C)C2=O)ccn1] 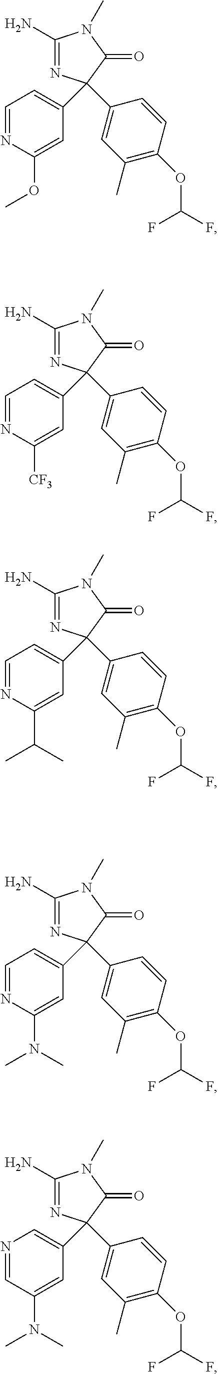 [SMILES: COc1cc(C2(c3ccc(OC(F)F)c(C)c3)N=C(N)N(C)C2=O)ccn1 ; Cc1cc(C2(c3ccnc(C(F)(F)F)c3)N=C(N)N(C)C2=O)ccc1OC(F)F ; Cc1cc(C2(c3ccnc(C(C)C)c3)N=C(N)N(C)C2=O)ccc1OC(F)F ; Cc1cc(C2(c3ccnc(N(C)C)c3)N=C(N)N(C)C2=O)ccc1OC(F)F ; Cc1cc(C2(c3cncc(N(C)C)c3)N=C(N)N(C)C2=O)ccc1OC(F)F] 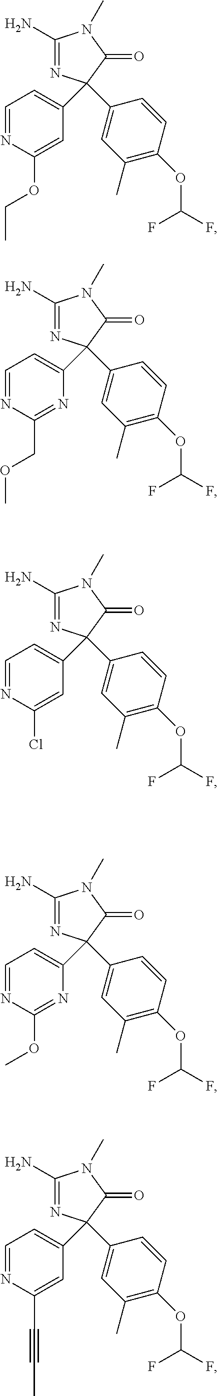 [SMILES: CCOc1cc(C2(c3ccc(OC(F)F)c(C)c3)N=C(N)N(C)C2=O)ccn1 ; COCc1nccc(C2(c3ccc(OC(F)F)c(C)c3)N=C(N)N(C)C2=O)n1 ; Cc1cc(C2(c3ccnc(Cl)c3)N=C(N)N(C)C2=O)ccc1OC(F)F ; COc1nccc(C2(c3ccc(OC(F)F)c(C)c3)N=C(N)N(C)C2=O)n1 ; CC#Cc1cc(C2(c3ccc(OC(F)F)c(C)c3)N=C(N)N(C)C2=O)ccn1] 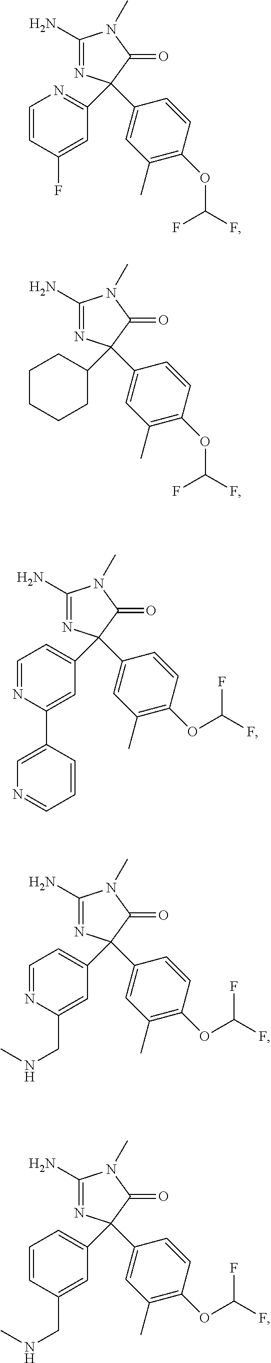 [SMILES: Cc1cc(C2(c3cc(F)ccn3)N=C(N)N(C)C2=O)ccc1OC(F)F ; Cc1cc(C2(C3CCCCC3)N=C(N)N(C)C2=O)ccc1OC(F)F ; Cc1cc(C2(c3ccnc(-c4cccnc4)c3)N=C(N)N(C)C2=O)ccc1OC(F)F ; CNCc1cc(C2(c3ccc(OC(F)F)c(C)c3)N=C(N)N(C)C2=O)ccn1 ; CNCc1cccc(C2(c3ccc(OC(F)F)c(C)c3)N=C(N)N(C)C2=O)c1] 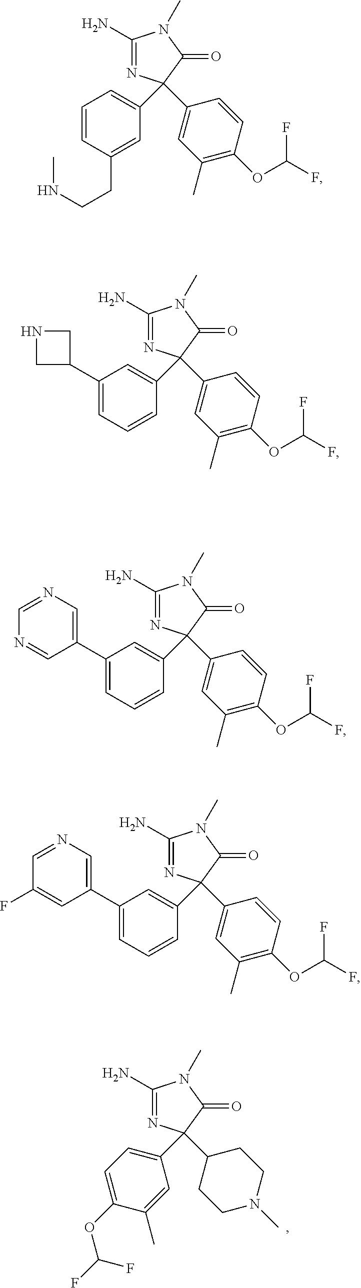 [SMILES: CNCCc1cccc(C2(c3ccc(OC(F)F)c(C)c3)N=C(N)N(C)C2=O)c1 ; Cc1cc(C2(c3cccc(C4CNC4)c3)N=C(N)N(C)C2=O)ccc1OC(F)F ; Cc1cc(C2(c3cccc(-c4cncnc4)c3)N=C(N)N(C)C2=O)ccc1OC(F)F ; Cc1cc(C2(c3cccc(-c4cncc(F)c4)c3)N=C(N)N(C)C2=O)ccc1OC(F)F ; Cc1cc(C2(C3CCN(C)CC3)N=C(N)N(C)C2=O)ccc1OC(F)F] 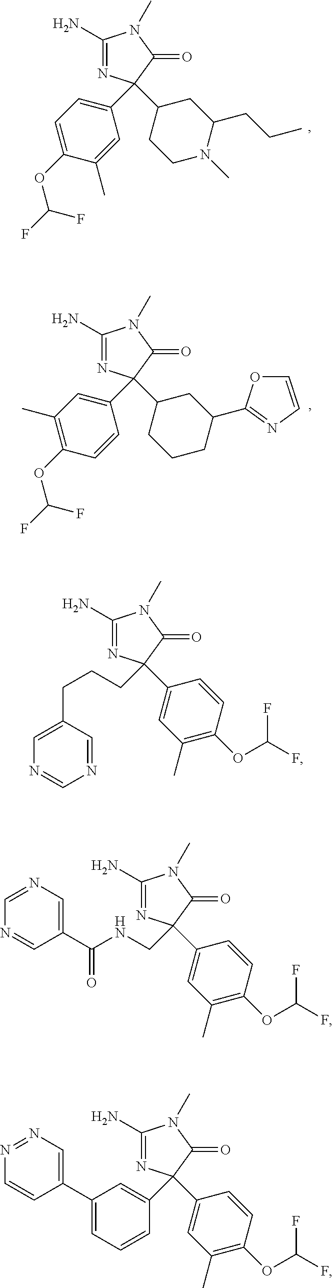 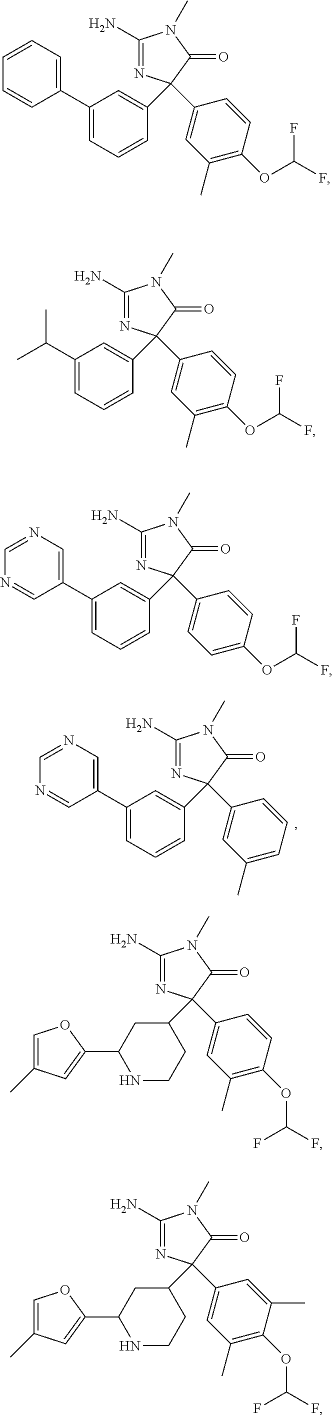 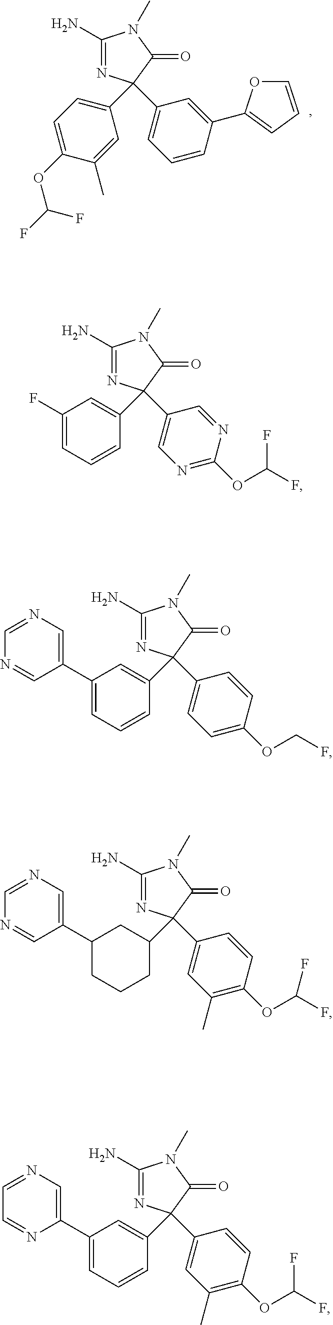 [SMILES: Cc1cc(C2(c3cccc(-c4ccco4)c3)N=C(N)N(C)C2=O)ccc1OC(F)F ; CN1C(=O)C(c2cnc(OC(F)F)nc2)(c2cccc(F)c2)N=C1N ; CN1C(=O)C(c2ccc(OCF)cc2)(c2cccc(-c3cncnc3)c2)N=C1N ; Cc1cc(C2(C3CCCC(c4cncnc4)C3)N=C(N)N(C)C2=O)ccc1OC(F)F ; Cc1cc(C2(c3cccc(-c4cnccn4)c3)N=C(N)N(C)C2=O)ccc1OC(F)F] 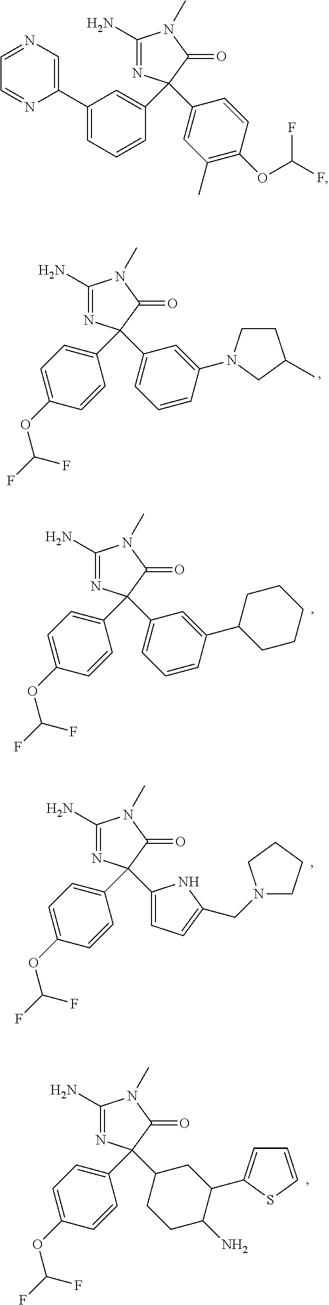 [SMILES: Cc1cc(C2(c3cccc(-c4cnccn4)c3)N=C(N)N(C)C2=O)ccc1OC(F)F ; CC1CCN(c2cccc(C3(c4ccc(OC(F)F)cc4)N=C(N)N(C)C3=O)c2)C1 ; CN1C(=O)C(c2ccc(OC(F)F)cc2)(c2cccc(C3CCCCC3)c2)N=C1N ; CN1C(=O)C(c2ccc(OC(F)F)cc2)(c2ccc(CN3CCCC3)[nH]2)N=C1N ; CN1C(=O)C(c2ccc(OC(F)F)cc2)(C2CCC(N)C(c3cccs3)C2)N=C1N] 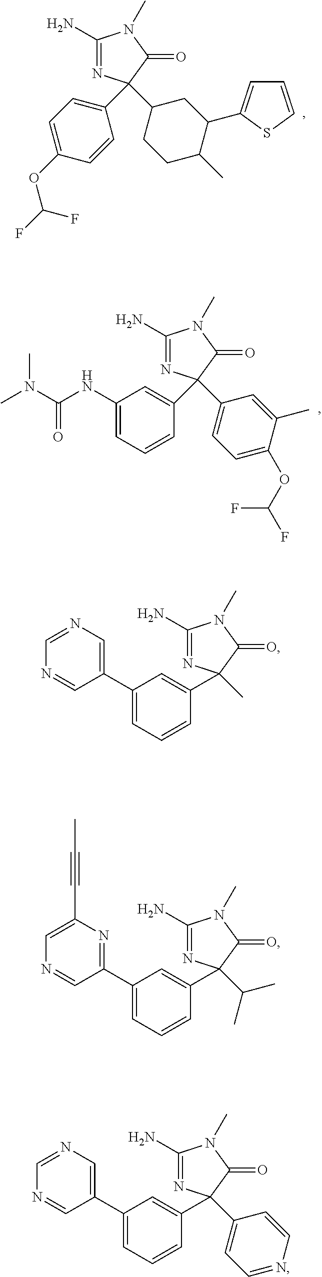 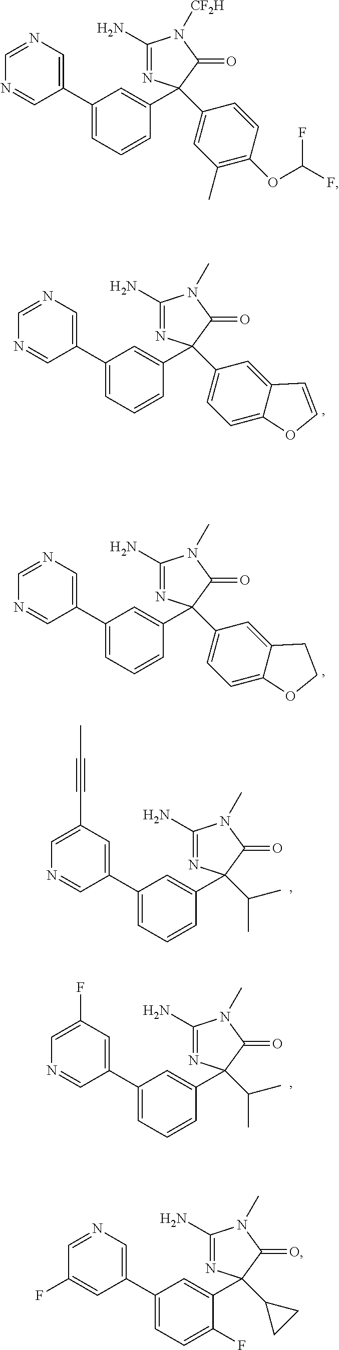 [SMILES: Cc1cc(C2(c3cccc(-c4cncnc4)c3)N=C(N)N(C(F)F)C2=O)ccc1OC(F)F ; CN1C(=O)C(c2cccc(-c3cncnc3)c2)(c2ccc3occc3c2)N=C1N ; CN1C(=O)C(c2cccc(-c3cncnc3)c2)(c2ccc3c(c2)CCO3)N=C1N ; CC#Cc1cncc(-c2cccc(C3(C(C)C)N=C(N)N(C)C3=O)c2)c1 ; CC(C)C1(c2cccc(-c3cncc(F)c3)c2)N=C(N)N(C)C1=O ; CN1C(=O)C(c2cc(-c3cncc(F)c3)ccc2F)(C2CC2)N=C1N] 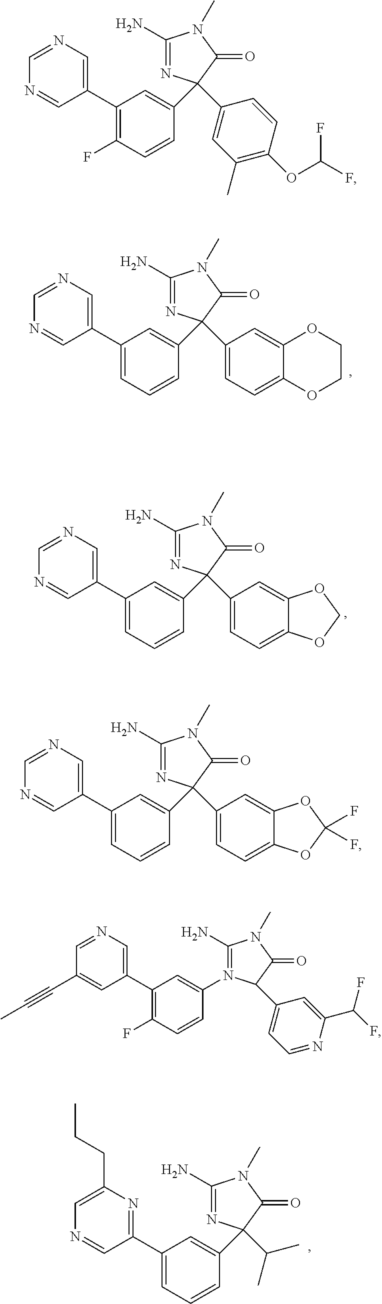 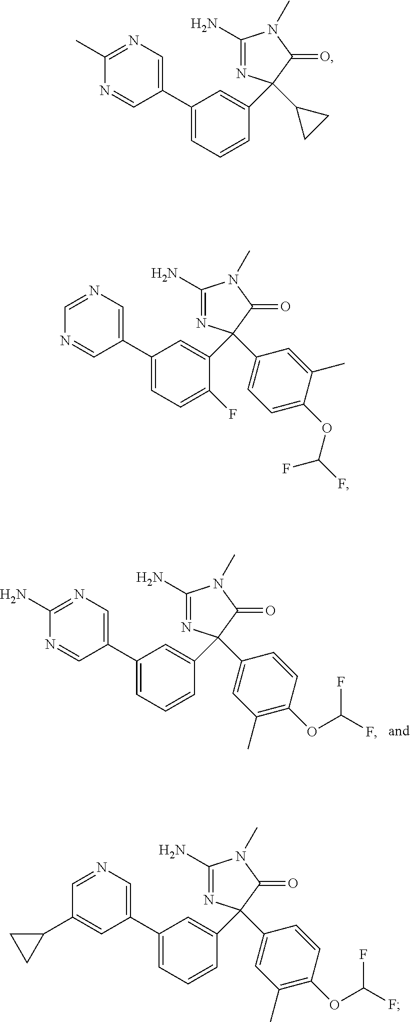 [SMILES: Cc1ncc(-c2cccc(C3(C4CC4)N=C(N)N(C)C3=O)c2)cn1 ; Cc1cc(C2(c3cc(-c4cncnc4)ccc3F)N=C(N)N(C)C2=O)ccc1OC(F)F ; Cc1cc(C2(c3cccc(-c4cnc(N)nc4)c3)N=C(N)N(C)C2=O)ccc1OC(F)F ; Cc1cc(C2(c3cccc(-c4cncc(C5CC5)c4)c3)N=C(N)N(C)C2=O)ccc1OC(F)F]
or a pharmaceutically acceptable salt thereof.

25. The compound of claim 1, having the structure: 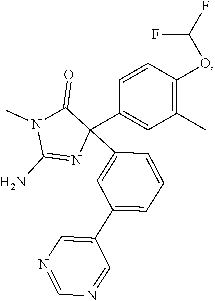 [SMILES: Cc1cc(C2(c3cccc(-c4cncnc4)c3)N=C(N)N(C)C2=O)ccc1OC(F)F]
or a pharmaceutically acceptable salt thereof.

26. The compound of claim 1, having the structure: 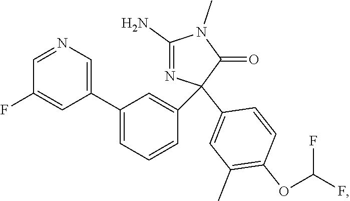 [SMILES: Cc1cc(C2(c3cccc(-c4cncc(F)c4)c3)N=C(N)N(C)C2=O)ccc1OC(F)F]
or a pharmaceutically acceptable salt thereof.

27. The compound of claim 1, having the structure: 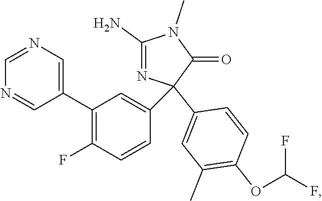 [SMILES: Cc1cc(C2(c3ccc(F)c(-c4cncnc4)c3)N=C(N)N(C)C2=O)ccc1OC(F)F]
or a pharmaceutically acceptable salt thereof.

28. A compound of claim 1, wherein the compound is selective for inhibition of BACE cleavage of APP relative to inhibition of BACE cleavage of NRG1 and/or PSGL1.

29. A pharmaceutical composition comprising a compound of any of claims 1-28 and a pharmaceutically acceptable excipient.

30. A method of treating Alzheimer's disease, comprising administering to a subject a compound of any of claims 1-28.

31. A method of treating Alzheimer's disease, comprising administering to a subject the pharmaceutical composition of claim 29.
